SEQUENCE LISTING (9 sequences)

SEQ ID NO: 1 - DNA, 3579 bp, Homo sapiens
  source 1..3579: mol_type = genomic DNA
atgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtga

SEQ ID NO: 2 - DNA, 810 bp, synthetic construct
  misc_feature 1..810: note = Synthetic Promoter
  source 1..810: mol_type = other DNA
gtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgccc

SEQ ID NO: 3 - DNA, 8562 bp, synthetic construct
  source 1..8562: mol_type = other DNA
gcccaatacgcaaaccgcctctccccgcgcgttggccgattcattaatgcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctggcgtaatagcgaagaggcccgcaccgatcgcccttcccaacagttgcgcagcctgaatggcgaatggcgattccgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaatcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagc

SEQ ID NO: 4 - DNA, 564 bp, synthetic construct
  misc_feature 1..564: note = Synthetic Promoter
  source 1..564: mol_type = other DNA
cagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 5 - DNA, 8409 bp, synthetic construct
  source 1..8409: mol_type = other DNA
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagacagccactatgggtctaggctgcccatgtaaggaggcaaggcctggggacacccgagatgcctggttataattaacccagacatgtggctgctccccccccccaacacctgctgcctgagcctcacccccaccccggtgcctgggtcttaggctctgtacaccatggaggagaagctcgctctaaaaataaccctgtccctggtgggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcaccaccacctccacagcacagacagacactcaggagccagccagccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 6 - DNA, 8611 bp, synthetic construct
  source 1..8611: mol_type = other DNA
gcccaatacgcaaaccgcctctccccgcgcgttggccgattcattaatgcagctggcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctaattatctacgtagccatgtctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgccagctggcgtaatagcgaagaggcccgcaccgatcgcccttcccaacagttgcgcagcctgaatggcgaatggaagttccagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgcgacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgatttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagagacctctcaaaaatagctaccctctccggcatgaatttatcagctagaacggttgaatatcatattgatggtgatttgactgtctccggcctttctcacccgtttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgttggaagttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcgtacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgggtttgagtgagctgataccgctcgccgcagccgaacgaccgagcgcagcgagtcagtgagcgaccaagcggaagagc

SEQ ID NO: 7 - DNA, 792 bp, synthetic construct
  misc_feature 1..792: note = Synthetic Promoter
  source 1..792: mol_type = other DNA
aagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagc

SEQ ID NO: 8 - DNA, 8629 bp, synthetic construct
  source 1..8629: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgattccaacatccaataaatcatacaggcaaggcaaagaattagcaaaattaagcaataaagcctcagagcataaagctaaatcggttgtaccaaaaacattatgaccctgtaatacttttgcgggagaagcctttatttcaacgcaaggataaaaatttttagaaccctcatatattttaaatgcaatgcctgagtaatgtgtaggtaaagattcaaacgggtgagaaaggccggagacagtcaaatcaccatcaatatgatattcaaccgttctagctgataaattcatgccggagagggtagctatttttgagaggtctctacaaaggctatcaggtcattgcctgagagtctggagcaaacaagagaatcgatgaacggtaatcgtaaaactagcatgtcaatcatatgtaccccggttgataatcagaaaagccccaaaaacaggaagattgtataagcaaatatttaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtataacgtgctttcctcgttagaatcagagcgggagctaaacaggaggccgattaaagggattttagacaggaacggtacgccagaatcctgagaagtgtttttataatcagtgaggccaccgagtaaaagagtctgtccatcacgcaaattaaccgttgtcgcaatacttctttgattagtaataacatcacttgcctgagtagaagaactcaaactatcggccttgctggtaatatccagaacaatattaccgccagccattgcaacggaatcgccattcgccattcaggctgcgcaactgttgggaagggcgatcggtgcgggcctcttcgctattacgccagct

SEQ ID NO: 9 - DNA, 4977 bp, synthetic construct
  source 1..4977: mol_type = other DNA
gcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctagagtttaaacaagcttgcatgtctaagctagacccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgtctagcatgccccactacgggtctaggctgcccatgtaaggaggcaaggcctggggacacccgagatgcctggttataattaacccagacatgtggctgcccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagcagccagcggcgcgcccaggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgccaccatgctgtggtgggaggaggtggaggattgttatgaaagggaggacgtgcagaagaagacttttaccaagtgggtgaacgctcagttcagcaaatttgggaagcagcacatcgagaatctgttttccgacctgcaggatgggagacggctgctggatctgctggaaggactgactggccagaagctgcccaaagagaaggggagcactagggtgcacgccctgaacaacgtgaacaaagctctgagagtgctgcagaacaacaacgtggatctggtgaatattggcagtactgatatcgtggacgggaaccacaaactgacactgggcctgatctggaacattattctgcactggcaggtgaaaaatgtgatgaagaacatcatggccgggctgcagcagaccaattccgagaagatcctgctgtcttgggtgcggcagagcacccgcaactatccccaggtgaacgtgattaacttcactacatcctggagcgacgggctggccctgaatgctctgattcacagccacaggcctgatctgttcgactggaatagcgtggtgtgccagcagtctgccacacagcgcctggaacatgccttcaatatcgctcggtaccagctggggatcgaaaaactgctggacccagaggatgtggacactacatacccagataaaaagtctattctgatgtacattactagcctgttccaggtgctgccacagcaggtgtctattgaagccattcaggaggtggaaatgctgccccgcccccccaaagtgactaaagaggagcattttcagctgcatcatcagatgcattacagccagcagattaccgtgagcctggctcagggatatgagcgcaccagtagtccaaaaccacggttcaagtcctacgcttatacccaggctgcctacgtgacaactagcgaccctactagatccccctttccatcccagcacctggaggccccagaggacaagagctttgggtccagcctgatggaaagcgaggtgaatctggatcggtaccagacagccctggaggaggtgctgagctggctgctgagtgctgaagacacactgcaggcccagggcgaaatttccaatgacgtggaagtggtgaaggatcagttccacacacacgagggctatatgatggacctgacagctcaccaggggcgcgtgggcaatatcctgcagctgggctctaaactgatcggcaccgggaaactgagtgaggacgaggaaacagaagtgcaggagcagatgaacctgctgaacagccgctgggagtgtctgagagtggctagtatggagaagcagtccaacctgcaccgggtgctgatggacctgcagaaccagaaactgaaagagctgaacgactggctgacaaagactgaggaacgcacaaggaagatggaggaggagccactgggacccgacctggaggatctgaagagacaggtgcagcagcataaggtgctgcaggaggatctggaacaggagcaggtgcgggtgaactccctgacacatatggtggtggtggtggacgaatctagtggagatcacgccaccgccgccctggaggaacagctgaaggtgctgggggaccggtgggccaacatttgccggtggaccgaggacaggtgggtgctgctgcaggacatcctgctgaaatggcagaggctgaccgaggagcagtgtctgtttagtgcttggctgagcgagaaagaggacgccgtgaacaagatccacacaaccggctttaaggatcagaacgaaatgctgtctagcctgcagaaactggctgtgctgaaggccgatctggagaaaaagaagcagagcatgggcaaactgtatagcctgaaacaggacctgctgagcaccctgaagaacaagagcgtgacccagaagacagaagcctggctggataactttgcccgctgctgggacaacctggtgcagaaactggagaaaagtacagctcagatctctcaggctgtgaccacaacccagcctagcctgacccagacaaccgtgatggaaaccgtgaccaccgtgacaacccgcgaacagatcctggtgaaacatgcccaggaagagctgccacctccacctccccagaagaagagaaccctggagcggctgcaggagctgcaggaagccactgacgaactggacctgaagctgaggcaggccgaagtgattaaggggtcttggcagcctgtgggcgatctgctgattgattccctgcaggaccacctggaaaaggtgaaggctctgagaggcgaaattgctccactgaaggagaacgtgagtcatgtgaacgatctggctagacagctgacaacactgggcatccagctgagcccatacaatctgagcacactggaggacctgaataccaggtggaagctgctgcaggtggctgtggaagaccgggtgcggcagctgcatgaggcccatcgcgacttcggaccagccagccagcactttctgagcacatccgtgcaggggccctgggagagggccatttctcccaacaaggtgccctactatattaatcacgagacccagaccacttgttgggaccatcccaagatgacagaactgtaccagtccctggccgatctgaacaacgtgaggtttagcgcttacagaaccgctatgaagctgagacggctgcagaaggccctgtgcctggatctgctgtccctgtccgccgcctgcgatgccctggatcagcataatctgaagcagaacgatcagccaatggatatcctgcagatcatcaactgcctgaccactatctacgacaggctggagcaggagcacaacaacctggtgaacgtgcctctgtgcgtggatatgtgcctgaactggctgctgaacgtgtatgacactgggcgcaccggccggatcagagtgctgagttttaaaactgggattatctccctgtgtaaggcccacctggaggacaagtacaggtacctgttcaagcaggtggctagtagcactggattttgtgaccagcgccgcctgggactgctgctgcatgatagtatccagattcctagacagctgggagaggtggctagtttcggaggatctaacatcgaacccagcgtgcgcagctgtttccagtttgccaataacaaacctgaaatcgaggctgctctgttcctggattggatgcgcctggaaccacagagcatggtgtggctgcctgtgctgcacagagtggctgccgccgaaactgccaagcaccaggctaaatgcaacatctgcaaggaatgtcccattatcggctttcgctacaggagtctgaaacattttaactacgatatttgccagagctgcttcttttccggaagagtggccaaaggacacaagatgcactaccctatggtggaatattgcaccccaactacatctggcgaagatgtgcgcgattttgccaaggtgctgaagaataagtttcggactaagaggtacttcgccaagcacccccgcatggggtatctgccagtgcagacagtgctggaaggagacaatatggagaccgatacaatgtgagcggccgcaataaaagatctttattttcattagatctgtgtgttggttttttgtgtgtctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgc

(19) United States
(12) Patent Application Publication
(10) Pub. No.: US 2026/0224751 A1
(43) Pub. Date: Aug. 6, 2026

(54) ADENO-ASSOCIATED VIRUS VECTOR DELIVERY OF MUSCLE SPECIFIC MICRO-DYSTROPHIN TO TREAT MUSCULAR DYSTROPHY

(71) Applicant: Research Institute at Nationwide Children's Hospital, Columbus, OH (US)

(72) Inventors: Louise Rodino-Klapac, Columbus, OH (US); Jerry R. Mendell, Columbus, OH (US)

(73) Assignee: Research Institute at Nationwide Children's Hospital, Columbus, OH (US)

(21) Appl. No.: 19/253,176

(22) Filed: Jun. 27, 2025

Related U.S. Application Data
(63) Continuation of application No. 17/253,956, filed on Jun. 17, 2019, now Pat. No. 12,491,265.
(60) Provisional application No. 62/686,668, filed on Jun. 18, 2018.
(60) Provisional application No. 62/740,402, filed on Oct. 2, 2018.
(60) Provisional application No. 62/752,841, filed on Oct. 30, 2018.
(60) Provisional application No. 62/823,649, filed on Mar. 25, 2019.
(60) Provisional application No. 62/860,220, filed on Jun. 11, 2019.

(51) Int. Cl. A61K 48/00; A61P 21/00; C07K 14/47; C12N 15/86

(52) U.S. Cl. CPC A61K 48/0058; A61K 48/0075; A61P 21/00; C07K 14/4716; C12N 15/86

(57) ABSTRACT

The invention provides gene therapy vectors, such as adeno-associated virus (AAV) vectors, expressing a miniaturized human micro-dystrophin gene and method of using these vectors to express micro-dystrophin in skeletal muscles including diaphragm and cardiac muscle and to protect muscle fibers from injury, increase muscle strength and reduce and/or prevent fibrosis in subjects suffering from muscular dystrophy.

### CROSS-REFERENCE TO RELATED APPLICATIONS

This application is a divisional of U.S. application Ser. No. 17/253,956, filed Dec. 18, 2020, now U.S. Pat. No. 12,491,265, which is a U.S. National Phase Application under 35 U.S.C. § 371 of International Application No. PCT/US2019/037489, filed Jun. 17, 2019, which claims priority benefit to U.S. Provisional Patent Application No. 62/686,668, filed Jun. 18, 2018; U.S. Provisional Patent Application No. 62/740,402, filed Oct. 2, 2018; U.S. Provisional Patent Application No. 62/752,841, filed Oct. 30, 2018; U.S. Provisional Patent Application No. 62/823,649, filed Mar. 25, 2019 and U.S. Provisional Patent Application No. 62/860,220 filed Jun. 11, 2018 each of which are incorporated by reference herein in their entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 140056-0328.xml; Size: 54,951 bytes, created; Dec. 28, 2025.

### FIELD OF INVENTION

The invention provides gene therapy vectors, such as adeno-associated virus (AAV) vectors, expressing a miniaturized human micro-dystrophin gene and method of using these vectors to express micro-dystrophin in skeletal muscles including diaphragm and cardiac muscle and to protect muscle fibers from injury, increase muscle strength and reduce and/or prevent fibrosis in subjects suffering from muscular dystrophy.

### BACKGROUND

The importance of muscle mass and strength for daily activities, such as locomotion and breathing, and for whole body metabolism is unequivocal. Deficits in muscle function produce muscular dystrophies (MDs) that are characterized by muscle weakness and wasting and have serious impacts on quality of life. The most well-characterized MDs result from mutations in genes encoding members of the dystrophin-associated protein complex (DAPC). These MDs result from membrane fragility associated with the loss of sarcolemmal-cytoskeleton tethering by the DAPC. Duchenne Muscular Dystrophy (DMD) is one of the most devastating muscle disease affecting 1 in 5000 newborn males.

DMD is caused by mutations in the DMD gene leading to reductions in mRNA and the absence of dystrophin, a 427 kD sarcolemmal protein associated with the dystrophin-associated protein complex (DAPC) (Hoffman et al., Cell 51 (6): 919-28, 1987). The DAPC is composed of multiple proteins at the muscle sarcolemma that form a structural link between the extra-cellular matrix (ECM) and the cytoskeleton via dystrophin, an actin binding protein, and alpha-dystroglycan, a laminin-binding protein. These structural links act to stabilize the muscle cell membrane during contraction and protect against contraction-induced damage. With dystrophin loss, membrane fragility results in sarcolemmal tears and an influx of calcium, triggering calcium-activated proteases and segmental fiber necrosis (Straub et al., Curr Opin. Neurol. 10 (2): 168-75, 1997). This uncontrolled cycle of muscle degeneration and regeneration ultimately exhausts the muscle stem cell population (Sacco et al., Cell, 2010. 143 (7): p. 1059-71; Wallace et al., Annu Rev Physiol, 2009. 71: p. 37-57), resulting in progressive muscle weakness, endomysial inflammation, and fibrotic scarring.

Without membrane stabilization from dystrophin or a micro-dystrophin, DMD will manifest uncontrolled cycles of tissue injury and repair ultimately replace lost muscle fibers with fibrotic scar tissue through connective tissue proliferation. Fibrosis is characterized by the excessive deposits of ECM matrix proteins, including collagen and elastin. ECM proteins are primarily produced from cytokines such as TGFβ that is released by activated fibroblasts responding to stress and inflammation. Although the primary pathological feature of DMD is myofiber degeneration and necrosis, fibrosis as a pathological consequence has equal repercussions. The over-production of fibrotic tissue restricts muscle regeneration and contributes to progressive muscle weakness in the DMD patient. In one study, the presence of fibrosis on initial DMD muscle biopsies was highly correlated with poor motor outcome at a 10-year follow-up (Desguerre et al., J Neuropathol Exp Neurol, 2009. 68 (7): p. 762-7). These results point to fibrosis as a major contributor to DMD muscle dysfunction and highlight the need for early intervention prior to overt fibrosis.

There is a need for treatments that increase muscle strength and protect against muscle injury in patients suffering from DMD.

### SUMMARY OF INVENTION

The present invention is directed to gene therapy vectors, e.g. AAV, expressing the micro-dystrophin gene to skeletal muscles including diaphragm and cardiac muscle to protect muscle fibers from injury, increase muscle strength and reduce and/or prevent fibrosis.

The invention provides for therapies and approaches for increasing muscular force and/or increasing muscle mass using gene therapy vectors to deliver micro-dystrophin to address the gene defect observed in DMD. Example 2 describes a systemic gene delivery clinical trial for Duchenne muscular dystrophy, in this study the subjects received 2×10¹⁴ vg/kg AAVrh74.MHCK7.micro-dystrophin. The clinical study described in Example 3 provides a novel pivotal clinical protocol that includes a randomized double-blind placebo controlled design. At study initiation, subjects are randomized and either receive 2×10¹⁴ vg/kg AAVrh74.MHCK7.micro-dystrophin or lactated ringers.

The invention provides for nucleic acid molecules comprising the nucleotide sequence of SEQ ID NO: 3, 8 or 9. The invention also provides for rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3.

Another aspect of the invention provides for compositions comprising a nucleic acid molecule comprising the nucleotide sequence of SEQ ID NO: 3, 8 or 9, rAAV comprising the nucleic acid sequence of SEQ ID NO: 9 or nucleotides 1-4977 of SEQ ID NO: 8 or nucleotides 55-5021 of SEQ ID NO: 3, and rAAV particles comprising the nucleic acid sequence of SEQ ID NO: 9 or nucleotides 1-4977 of SEQ ID NO: 8 or nucleotides 55-5021 of SEQ ID NO: 3. Any of the methods disclosed herein may be carried out with these compositions.

The invention provides for methods of treating a muscular dystrophy in a human subject in need thereof comprising the step of administering a recombinant adeno-virus associated (rAAV) rAAV.MHCK7.microdystrophin, wherein the rAAV is administered by a systemic route of administration at a dose of about 5.0×10¹² vg/kg to about 1.0×10¹⁵ vg/kg. The muscular dystrophy may be Duchenne muscular dystrophy or Becker's muscular dystrophy.

For example, the dose of rAAV administered is about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg.

In one embodiment, the methods of the invention comprise systemically administering rAAV wherein the systemic route of administration is an intravenous route and the dose of the rAAV administered is about 2.0×10¹⁴ vg/kg. In another embodiment, the methods of the invention comprise systemically administering rAAV wherein the systemic route of administration is an intravenous route and the dose of the rAAV administered is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one embodiment, the rAAV is AAVrh74.MHCK7.microdystrophin or AAVrh74.MCK.microdystrophin. In one embodiment, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is the AAVrh74.MCK.microdystrophin of nucleotides 56-4820 of SEQ ID NO: 5.

In any of the methods of the invention, the dose of rAAV can be administered at about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular embodiment, the dose or the rAAV is administered in about 10 mL/kg. In one embodiment, the rAAV is AAVrh74.MHCK7.microdystrophin or AAVrh74.MCK.microdystrophin. In one embodiment, the rAAV is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is the AAVrh74.MCK.microdystrophin of nucleotides 56-4820 of SEQ ID NO: 5.

In any of the methods of the invention, the dose of rAAV can be administered by injection, infusion or implantation. For example, the dose of rAAV is administered by infusion over approximately one hour. In addition, the dose of rAAV is administered by an intravenous route through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8 or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The rAAV administered by any of the methods of the invention can comprise the human micro-dystrophin nucleotide sequence of SEQ ID NO: 1, the MHCK7 promoter sequence of SEQ ID NO: 2 or SEQ ID NO:7. In addition, the rAAV administered by any of the methods of the invention comprises the human micro-dystrophin nucleotide sequence of SEQ ID NO: 1 and the MHCK7 promoter sequence of SEQ ID NO: 2 or SEQ ID NO:7. For example, the rAAV can comprise the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6.

In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the methods of the invention, the rAAV administered is of the serotype AAVrh7.4.

In some embodiments, the methods of the invention treat Duchenne muscular dystrophy or Becker's muscular dystrophy. An exemplary embodiment is a method of treating Duchenne muscular dystrophy or Becker's muscular dystrophy in a human subject in need thereof comprising the step of administering a dose recombinant adeno-virus associated (rAAV) rAAV.MHCK7.microdystrophin, wherein the route of administration is intravenous infusion and the dose of the rAAV administered is about 2×10¹⁴ vg/kg over approximately one hour, and wherein the rAAV vector comprises the AAVrh74.MHCK7.micro-dystrophin construct nucleotide sequence of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the invention provides for a rAAV comprising a muscle specific control element nucleotide sequence, and a nucleotide sequence encoding the micro-dystrophin protein. For example, the nucleotide sequence encodes a functional micro-dystrophin protein, wherein the nucleotide has, e.g., at least 65%, at least 70%, at least 75%, at least 80%, 81%, 82%, 83%, 84%, 85%, 86%, 87%, 88%, or 89%, more typically at least 90%, 91%, 92%, 93%, or 94% and even more typically at least 95%, 96%, 97%, 98%, 99%, or 100% sequence identity to SEQ ID NO: 1, wherein the protein retains micro-dystrophin activity. The micro-dystrophin protein provides stability to the muscle membrane during muscle contraction, e.g. micro-dystrophin acts as a shock absorber during muscle contraction. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9 or of nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The invention also provides for rAAV wherein the nucleotide sequence comprises a nucleotide sequence that hybridizes under stringent conditions to the nucleic acid sequence of SEQ ID NO: 1, or compliments thereof, and encodes a functional micro-dystrophin protein.

In one embodiment, the rAAV is a non-replicating, recombinant adeno-associated virus (AAV) termed AAVrh74.MHCK7.micro-dystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. This vector genome contains minimal elements required for gene expression, including AAV2 inverted terminal repeats (ITR), the micro-dystrophin, SV40 intron (SD/SA), and synthetic polyadenylation (Poly A) signal, all under the control of the MHCK7 promoter/enhancer. The schematic of the vector genome and expression cassette is shown FIG. 1. The AAVrh74 serotype can be employed to achieve efficient gene transfer in skeletal and cardiac muscle following IV administration.

The term “stringent” is used to refer to conditions that are commonly understood in the art as stringent. Hybridization stringency is principally determined by temperature, ionic strength, and the concentration of denaturing agents such as formamide. Examples of stringent conditions for hybridization and washing are 0.015 M sodium chloride, 0.0015 M sodium citrate at 65-68° C. or 0.015 M sodium chloride, 0.0015M sodium citrate, and 50% formamide at 42° C. See Sambrook et al., Molecular Cloning: A Laboratory Manual, 2nd Ed., Cold Spring Harbor Laboratory, (Cold Spring Harbor, N.Y. 1989). More stringent conditions (such as higher temperature, lower ionic strength, higher formamide, or other denaturing agent) may also be used, however, the rate of hybridization will be affected. In instances wherein hybridization of deoxyoligonucleotides is concerned, additional exemplary stringent hybridization conditions include washing in 6×SSC 0.05% sodium pyrophosphate at 37° C. (for 14-base oligos), 48° C. (for 17-base oligos), 55° C. (for 20-base oligos), and 60° C. (for 23-base oligos).

Other agents may be included in the hybridization and washing buffers for the purpose of reducing non-specific and/or background hybridization. Examples are 0.1% bovine serum albumin, 0.1% polyvinyl-pyrrolidone, 0.1% sodium pyrophosphate, 0.1% sodium dodecylsulfate, NaDodSO4, (SDS), ficoll, Denhardt's solution, sonicated salmon sperm DNA (or other non-complementary DNA), and dextran sulfate, although other suitable agents can also be used. The concentration and types of these additives can be changed without substantially affecting the stringency of the hybridization conditions. Hybridization experiments are usually carried out at pH 6.8-7.4, however, at typical ionic strength conditions, the rate of hybridization is nearly independent of pH. See Anderson et al., Nucleic Acid Hybridisation: A Practical Approach, Ch. 4, IRL Press Limited (Oxford, England). Hybridization conditions can be adjusted by one skilled in the art in order to accommodate these variables and allow DNAs of different sequence relatedness to form hybrids.

The term “muscle specific control element” refers to a nucleotide sequence that regulates expression of a coding sequence that is specific for expression in muscle tissue. These control elements include enhancers and promoters. The invention provides for constructs comprising the muscle specific control elements MCKH7 promoter, the MCK promoter and the MCK enhancer.

The term “operably linked” refers to the positioning of the regulatory element nucleotide sequence, e.g. promoter nucleotide sequence, to confer expression of said nucleotide sequence by said regulatory element.

In one aspect, the invention provides for a rAAV wherein the muscle specific control element is a human skeletal actin gene element, cardiac actin gene element, myocyte-specific enhancer binding factor (MEF), muscle creatine kinase (MCK), truncated MCK (tMCK), myosin heavy chain (MHC), hybrid α-myosin heavy chain enhancer-/MCK enhancer-promoter (MHCK7), C5-12, murine creatine kinase enhancer element, skeletal fast-twitch troponin c gene element, slow-twitch cardiac troponin c gene element, the slow-twitch troponin i gene element, hypoxia-inducible nuclear factors, steroid-inducible element or glucocorticoid response element (GRE).

For example, the muscle specific control element is the MHCK7 promoter nucleotide sequence SEQ ID NO: 2 or SEQ ID NO: 7, or the muscle specific control element is MCK nucleotide sequence SEQ ID NO: 4. In addition, in any of the rAAV vectors of the invention, the muscle specific control element nucleotide sequence, e.g. MHCK7 or MCK nucleotide sequence, is operably linked to the nucleotide sequence encoding the micro-dystrophin protein. For example, the MHCK7 promoter nucleotide sequence (SEQ ID NO: 2 or SEQ ID NO: 7) is operably linked to the human micro-dystrophin coding sequence (SEQ ID NO: 1) as set out in the construct provided in FIG. 1 or FIG. 2 (SEQ ID NO: 3) or FIG. 13 (SEQ ID NO: 9). In another example, the MCK promoter (SEQ ID NO: 4) is operably linked to the human micro-dystrophin coding sequence (SEQ ID NO: 1) as set out in the construct provided in FIG. 5 or FIG. 6 (SEQ ID NO: 5). In another aspect, the invention provides for a rAAV vector comprising the nucleotide sequence of SEQ ID NO: 1 and SEQ ID NO: 2, or SEQ ID NO: 1 and SEQ ID NO: 7. The invention also provides for a rAAV vector comprising the nucleotide sequence of SEQ ID NO: 1 and SEQ ID NO: 4.

In a further aspect, the invention provides for an rAAV construct contained in the plasmid comprising the nucleotide sequence of SEQ ID NO: 3, SEQ ID NO: 5, SEQ ID NO: 6, or SEQ ID NO: 8. For example, the AAVrh74.MHCK7.microdystrophin vector comprises the nucleotide sequence within and inclusive of the ITR's of SEQ ID NO: 3 and shown in FIG. 2. The rAAV vector comprises the 5′ ITR, MHCK7 promoter, a chimeric intron sequence, the coding sequence for the human micro-dystrophin gene, polyA, and 3′ ITR. In one embodiment, the vector comprises nucleotides 55-5021 of SEQ ID NO:3. The plasmid set forth in SEQ ID NO:3 further comprises ampicillin resistance and the pGEX plasmid backbone with pBR322 origin of replication.

In another aspect, the invention provides for a rAAV comprising the nucleotide sequence of SEQ ID NO: 9. For example, the AAVrh74.MHCK7.microdystrophin vector construct comprises the nucleotide sequence of SEQ ID NO: 9 and shown in FIG. 13. This rAAV vector construct comprises the MHCK7 promoter, a chimeric intron sequence, the coding sequence for the human micro-dystrophin gene, and polyA. In one embodiment, the rAAV vector construct further comprises an ITR 5′ to the promoter, and an ITR 3′ to the polyA. In one embodiment, the rAAV is AAVrh74.

In another aspect, the AAVrh74.MHCK7.microdystrophin vector comprises the nucleotide sequence within and inclusive of the ITR's of SEQ ID NO: 8 and shown in FIG. 15. The rAAV vector comprises the 5′ ITR, MHCK7 promoter, a chimeric intron sequence, the coding sequence for the human micro-dystrophin gene, polyA, and 3′ ITR. In one embodiment, the vector comprises nucleotides 1-4977 of SEQ ID NO:9. The plasmid set forth in SEQ ID NO:3 further comprises kanamycin resistance and the pGEX plasmid backbone with pBR322 origin of replication.

In another aspect, the invention provides for a plasmid comprising the AAVrh74.MHCK7.microdystrophin vector construct. In one embodiment, the plasmid comprises the 5′ ITR, MHCK7 promoter, a chimeric intron sequence, the coding sequence for the human micro-dystrophin gene, polyA, and 3′ ITR. In one embodiment, the plasmid comprises kanamycin resistance and optionally comprises the pGEX plasmid backbone with pBR322 origin of replication. In a particular embodiment, the plasmid is set forth in SEQ ID NO:8, and shown in FIGS. 14 and 15.

The invention provides for a recombinant AAV vector comprising the human micro-dystrophin nucleotide sequence of SEQ ID NO: 1 and the MHCK7 promoter nucleotide sequence of SEQ ID NO: 2 or SEQ ID NO:7. This rAAV vector is the AAV serotype AAVrh.74.

The invention also provides for a rAAV comprising the AAVrh74.MHCK7.micro-dystrophin construct nucleotide sequence within and inclusive of the ITR's in SEQ ID NO: 3, the nucleotide sequence within and inclusive of the ITR's in SEQ ID NO: 8 or the nucleotide sequence as set forth in SEQ ID NO: 9. This rAAV vector is the AAV serotype AAVrh.74.

The rAAV vectors of the invention may be any AAV serotype, such as the serotype AAVrh. 74, AAV1, AAV2, AAV4, AAV5, AAV6, AAV7, AAV8, AAV9, AAV10, AAV11, AAV12 or AAV13.

The invention also provides for pharmaceutical compositions (or sometimes referred to herein as simply “compositions”) comprising any of the rAAV vectors of the invention.

In another embodiment, the invention provides for methods of producing a rAAV vector particle comprising culturing a cell that has been transfected with any rAAV vector of the invention and recovering rAAV particles from the supernatant of the transfected cells. The invention also provides for viral particles comprising any of the recombinant AAV vectors of the invention.

In any of the methods of treating a muscular dystrophy, the level of micro-dystrophin gene expression in a cell of the subject is increased after administration of the rAAV. Expression of the micro-dystrophin gene in the cell is detected by measuring the micro-dystrophin protein level by Western blot in muscle biopsied before and after administration of the rAAV. In particular, the level of micro-dystrophin protein is increased by at least about 70% to at least about 80%, or at least about 70% to at least about 90%, or at least about 80% to at least about 90% after administration of rAAV compared to the level of micro-dystrophin before administration of rAAV. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of micro-dystrophin before administration of rAAV.

In addition, expression of the micro-dystrophin gene in the cell is detected by measuring the micro-dystrophin protein level by immunohistochemistry in muscle biopsies before and after administration of the rAAV. The level of micro-dystrophin protein is increased by at least about 70% to at least about 80%, or at least about 70% to at least about 90%, or at least about 80% to at least about 90% after administration of rAAV compared to the level of micro-dystrophin before administration of rAAV. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of rAAV compared to the level of micro-dystrophin before administration of rAAV.

In any of the methods of treating a muscular dystrophy, the serum CK level in the subject is decreased after administration of the rAAV as compared to serum CK level before administration of the rAAV. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, in any of the methods of treating a muscular dystrophy of the invention, the serum CK level in the subject is decreased by about 87% by 60 days after administration of the rAAV as compared to the serum CK level before administration of the rAAV or in any of the methods of treating a muscular dystrophy of the invention, the serum CK level in the subject is decreased by about 72% by 60 days after administration of the rAAV as compared to the serum CK level before administration of the rAAV, or in any of the methods of treating a muscular dystrophy of the invention, the serum CK level in the subject is decreased by about 73% by 60 days after administration of the rAAV as compared to the serum CK level before administration of the rAAV, or in any of the methods of treating a muscular dystrophy of the invention, the serum CK level in the subject is decreased by about 78% by 60 days after administration of the rAAV as compared to the serum CK level before administration of the rAAV. or in any of the methods of treating a muscular dystrophy of the invention, the serum CK level in the subject is decreased by about 95% by 60 days after administration of the rAAV as compared to the serum CK level before administration of the rAAV. In any of the methods of treating a muscular dystrophy, the number of micro-dystrophin positive fibers in the muscle tissue of the subject is increased after administration of the rAAV as compared to the number of micro-dystrophin positive fibers before administration of the rAAV. For example, the number of micro-dystrophin positive fibers is detected by measuring the micro-dystrophin protein level by Western blot or immunohistochemistry on muscle biopsies before and after administration of the rAAV.

In any of the methods of treating a muscular dystrophy, administration of the rAAV upregulates expression of DAPC proteins such as alpha-sarcoglycan or beta-sarcoglycan. For example, the level of alpha-sarcoglycan in the subject is increased after administration of the rAAV as compared to the level of alpha-sarcoglycan before administration of the rAAV. In addition, the level of beta-sarcoglycan in the subject is increased after administration of the rAAV as compared to the level of the beta-sarcoglycan before administration of the rAAV. The level of alpha-sarcoglycan or beta-sarcoglycan is detected by measuring the alpha-sarcoglycan or beta-sarcoglycan protein level by Western blot or immunohistochemistry on muscle biopsies before and after administration of the rAAV.

In any of the methods of the treating a muscular dystrophy, disease progression in the subject is delayed after administration of the rAAV as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, the subject has at least a 6-point improvement in NSAA score at least 270 days after administration of the rAAV as compared to NSAA score before administration of the rAAV. Further, in any of the methods, the subject has at least about 0.8 second improvement in time to rise at least 270 days after administration of the rAAV as compared to time to rise before administration of the rAAV. In addition, in any of the methods, the subject has at least about 1.2 second improvement in time to ascend 4 steps test at least 270 days after administration of the rAAV as compared to time to ascend 4 steps test before administration of the rAAV. In addition, in any of the methods, the subject has at least about 7 second improvement in the 100 m timed test at least 270 days after administration of the rAAV as compared to the 100 m timed test before administration of the rAAV.

In another embodiment, the invention provides for methods of expressing micro-dystrophin gene in a patient cell comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. For example, expression of the micro-dystrophin gene in the patient cell is detected by measuring the micro-dystrophin protein level by Western blot or immunohistochemistry in muscle biopsies before and after administration of the rAAV.MHCK7.micro-dystrophin construct. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In a further embodiment, the invention provides for methods of decreasing serum CK levels in a patient in need thereof, the method comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the rAAV as compared to the serum CK level before administration of the rAAV. In particular, the serum CK level in the subject is decreased by about 87% by 60 days after administration of the rAAV as compared to the serum CK level before administration of the rAAV or in any of the methods of treating a muscular dystrophy of the invention, the serum CK level in the subject is decreased by about 72% by 60 days after administration of the rAAV as compared to the serum CK level before administration of the rAAV, or in any of the methods of treating a muscular dystrophy of the invention, the serum CK level in the subject is decreased by about 73% by 60 days after administration of the rAAV as compared to the serum CK level before administration of the rAAV, or in any of the methods of treating a muscular dystrophy of the invention, the serum CK level in the subject is decreased by about 78% by 60 days after administration of the rAAV as compared to the serum CK level before administration of the rAAV, or in any of the methods of treating a muscular dystrophy of the invention, the serum CK level in the subject is decreased by about 95% by 60 days after administration of the rAAV as compared to the serum CK level before administration of the rAAV.

The invention also provides for methods of increasing micro-dystrophin positive fibers in a patient muscle tissue comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the number of micro-dystrophin positive fibers is detected by measuring the dystrophin protein level by Western blot or immunohistochemistry on muscle biopsies before and after administration of the rAAV. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another embodiment, the invention provides for methods of increasing the expression of alpha-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of alpha-sarcoglycan is detected by measuring the alpha-sarcoglycan protein level by Western blot or immunohistochemistry on muscle biopsies before and after administration of the rAAV.

In addition, the invention provides for methods of increasing the expression of beta-sarcoglycan in a patient in need thereof comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, the level of beta-sarcoglycan is detected by measuring the beta-sarcoglycan protein level by Western blot or immunohistochemistry on muscle biopsies before and after administration of the rAAV.

The invention also provides for methods of treating a patient with Duchenne muscular dystrophy or Becker muscular dystrophy comprising administering to the patient the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6, such that disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, in any of the methods, the subject has at least a 6-point improvement in NSAA score at least 270 days after administration of the rAAV as compared to NSAA score before administration of the rAAV. Further, in any of the methods, the subject has at least about 0.8 second improvement in time to rise at least 270 days after administration of the rAAV as compared to time to rise before administration of the rAAV. In addition, in any of the methods, the subject has at least about 1.2 second improvement in time to ascend 4 steps test at least 270 days after administration of the rAAV as compared to time to ascend 4 steps test before administration of the rAAV. In addition, in any of the methods, the subject has at least about 7 second improvement in the 100 m timed test at least 270 days after administration of the rAAV as compared to the 100 m timed test before administration of the rAAV.

“Fibrosis” refers to the excessive or unregulated deposition of extracellular matrix (ECM) components and abnormal repair processes in tissues upon injury, including skeletal muscle, cardiac muscle, liver, lung, kidney, and pancreas. The ECM components that are deposited include fibronectin and collagen, e.g. collagen 1, collagen 2 or collagen 3.

The invention also provides for methods of reducing or preventing fibrosis in a subject suffering from muscular dystrophy comprising administering a therapeutically effective amount of a rAAV comprising the human micro-dystrophin nucleotide sequence of SEQ ID NO: 1 and the MHCK7 promoter nucleotide sequence of SEQ ID NO: 2 or SEQ ID NO: 7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MHCK7.microdystrophin. In one embodiment, the AAVrh74.MHCK7.microdystrophin is the AAVrh74.MHCK7.microdystrophin of nucleotides 55-5021 of SEQ ID NO: 3. In another embodiment, the AAVrh74.MHCK7.microdystrophin is the AAVrh74.MHCK7.microdystrophin of SEQ ID NO: 9. In another embodiment, the AAVrh74.MHCK7.microdystrophin is the AAVrh74.MHCK7.microdystrophin of nucleotides 1-4977 of SEQ ID NO: 8 or nucleotides 56-5066 of SEQ ID NO: 6. In a further embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In another embodiment, the invention provides for methods of preventing fibrosis in a subject in need thereof, comprising administering a therapeutically effective amount of the human micro-dystrophin nucleotide sequence of SEQ ID NO: 1 and the MHCK7 promoter nucleotide sequence of SEQ ID NO: 2 or SEQ ID NO:7; or rAAV vector comprising the AAV74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. For example, any of the rAAV of the invention can be administered to subjects suffering from muscular dystrophy to prevent fibrosis, e.g. the rAAV of the invention expressing a human micro-dystrophin protein administered before fibrosis is observed in the subject. In addition, the rAAV of the invention expressing a human micro-dystrophin gene can be administered to a subject at risk of developing fibrosis, such as those suffering or diagnosed with muscular dystrophy, e.g. DMD. The rAAV of the invention can be administered to the subject suffering from muscular dystrophy in order to prevent new fibrosis in these subjects.

The invention contemplates administering rAAV before fibrosis is observed in the subject. In addition, the rAAV can be administered to a subject at risk of developing fibrosis, such as those suffering or diagnosed with a muscular dystrophy, e.g. DMD. The rAAV can be administered to the subject suffering from muscular dystrophy who already has developed fibrosis in order to prevent new fibrosis in these subjects.

The invention also provides for methods of increasing muscular force and/or muscle mass in a subject suffering from a muscular dystrophy comprising administering a therapeutically effective amount of the human micro-dystrophin nucleotide sequence of SEQ ID NO: 1 and the MHCK7 promoter nucleotide sequence of SEQ ID NO: 2 or SEQ ID NO: 7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

The invention contemplates administering rAAV vectors to subjects diagnosed with DMD before fibrosis is observed in the subject or before the muscle force has been reduced or before the muscle mass has been reduced.

The invention also contemplates administering the human micro-dystrophin nucleotide sequence of SEQ ID NO: 1 and the MHCK7 promoter nucleotide sequence of SEQ ID NO: 2 or SEQ ID NO:7; or a rAAV comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to a subject suffering from a muscular dystrophy who already has developed fibrosis, in order to prevent new fibrosis in these subjects or to reduce fibrosis in these subjects. The invention also provides for administering the human micro-dystrophin nucleotide sequence of SEQ ID NO: 1 and the MHCK7 promoter nucleotide sequence of SEQ ID NO: 2 or SEQ ID NO:7; or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 to the subject suffering from a muscular dystrophy who already has reduced muscle force or has reduced muscle mass in order to protect the muscle from further injury.

In any of the methods of the invention, the subject may be suffering from a muscular dystrophy such as DMD or any other dystrophin-associated muscular dystrophy.

In other embodiments of any of the methods of the invention described herein, the serum CK level in the subject is decreased after administration of the rAAV as compared to the serum CK level before administration of the rAAV by a percentage level selected from the group consisting of:
a) at least 78% by 90, 180, or 270 days after the administration;
b) at least 46, 55, 56, 57, 58, 59, 60, 61, 62, 63, 64, 65, 66, 67, 68, 69, 70 or 85% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) at least 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another embodiment, the invention provides for compositions for treating a muscular dystrophy in a human subject in need, wherein the composition comprises a dose of recombinant adeno-virus associated (rAAV) rAAV.MHCK7.microdystrophin, wherein composition is formulated for a systemic route of administration and the dose of the rAAV is about 1×10¹⁴ vg/kg to about 4×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

For example, the composition of the invention comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the compositions of the invention are formulated for intravenous administration and comprise a dose of rAAV that is about 2.0×10¹⁴ vg/kg. In another embodiment, the compositions of the invention are formulated for intravenous administration and comprise a dose of rAAV that is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the compositions of the invention, the dose of rAAV is delivered in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/kg, or about 10 mL/kg to 15 mL/kg or 10 mL/kg to about 20 mL/kg. In a particular embodiment, the composition comprises a dose of the rAAV delivered in about 10 mL/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In another embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

The compositions of the invention are formulated for administration by injection, infusion or implantation. For example, the compositions are formulated for administration by infusion over approximately one hour. In addition, the compositions of the invention are formulated for intravenous administration through a peripheral limb vein such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

Any of the compositions of the invention comprise a rAAV comprising the human micro-dystrophin nucleotide sequence of SEQ ID NO: 1 and the MHCK7 promoter sequence of SEQ ID NO: 2 or SEQ ID NO:7 or a rAAV vector comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In particular, the compositions of the invention are for treating Duchenne muscular dystrophy or Becker's muscular dystrophy. For example, the invention provides for compositions for treating Duchenne muscular dystrophy or Becker's muscular dystrophy in a human subject in need thereof wherein the composition comprises a dose of recombinant adeno-virus associated (rAAV) rAAV.MHCK7.microdystrophin, wherein the composition is formulated for administration by intravenous infusion over approximately one hour and the dose of the rAAV administered is about 2×10¹⁴ vg/kg, and wherein the rAAV comprises the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In another embodiment, the invention also provides a composition comprising rAAV for reducing fibrosis in a subject in need thereof. In addition, the invention provides a composition comprising a rAAV vectors for preventing fibrosis in a subject suffering from a muscular dystrophy.

The invention also provides for compositions comprising rAAV for increasing muscular force and/or muscle mass in a subject suffering from a muscular dystrophy. In a further embodiment, the invention provides for compositions comprising any of the rAAV of the invention for treatment of muscular dystrophy.

In other embodiments of any of the compositions of the invention, after administration of said composition to a human subject in need of treatment for muscular dystrophy, the serum CK level in the subject is decreased as compared to the serum CK level before administration of the composition by a percentage level selected from the group consisting of:
a) at least 78% by 90, 180, or 270 days after the administration;
b) at least 46, 55, 70, or 85% by 270 days after the administration;
c) at least 72, 73, 74, or 95% by 180 days after the administration;
d) at least 87, 99, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In another embodiment, the invention provides for use of a dose of recombinant adeno-virus associated (rAAV) rAAV.MHCK7.microdystrophin for the preparation of a medicament for the treatment of muscular dystrophy in a human subject in need thereof, wherein the medicament is formulated for a systemic route of administration and the dose of the rAAV is about 1×10¹⁴ vg/kg to about 4×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

For example, the medicament comprises a dose of rAAV of about 5.0×10¹² vg/kg to about 1.0×10¹⁴ vg/kg, or about 5.0×10¹² vg/kg to 1.0×10¹⁴ vg/kg, or about 5.0×10¹² vg/kg to about 2.0×10¹⁴ vg/kg, or about 5.0×10¹² vg/kg to about 1.0×10¹⁴ vg/kg, or about 5.0×10¹² vg/kg to about 5.0×10¹³ vg/kg, or about 5.0×10¹² vg/kg to about 2.0×10¹³ vg/kg, or about 5.0×10¹² vg/kg to about 1.0×10¹³ vg/kg, or 1.0×10¹⁴ vg/kg to about 1.0×10¹⁵ vg/kg, or 1.0×10¹³ vg/kg to about 1.0×10¹⁴ vg/kg, or about 1.0×10¹³ vg/kg to 1.0×10¹⁴ vg/kg, or about 1.0×10¹³ vg/kg to about 2.0×10¹⁴ vg/kg, or about 1.0×10¹³ vg/kg to about 1.0×10¹⁴ vg/kg, or about 1.0×10¹³ vg/kg to about 5.0×10¹³ vg/kg, or about 1.0×10¹³ vg/kg to about 3.0×10¹⁴ vg/kg, or about 1.0×10¹³ vg/kg to about 5.0×10¹⁴ vg/kg, or about 1.0×10¹³ vg/kg to about 6.0×10¹⁴ vg/kg, or 1.0×10¹³ vg/kg to about 1.0×10¹⁵ vg/kg, or 5.0×10¹³ vg/kg to about 1.0×10¹⁴ vg/kg, or about 5.0×10¹³ vg/kg to 1.0×10¹⁴ vg/kg, or about 5.0×10¹³ vg/kg to about 2.0×10¹⁴ vg/kg, or about 5.0×10¹³ vg/kg to about 1.0×10¹⁴ vg/kg, or about 5.0×10¹³ vg/kg to about 3.0×10¹⁴ vg/kg, or about 5.0×10¹³ vg/kg to about 5.0×10¹⁴ vg/kg, or about 5.0×10¹³ vg/kg to about 6.0×10¹⁴ vg/kg, or 5.0×10¹³ vg/kg to about 1.0×10¹⁵ vg/kg, or 1.0×10¹⁴ vg/kg to about 6.0×10¹⁴ vg/kg, or 1.0×10¹⁴ vg/kg to about 5.0×10¹⁴ vg/kg, or 1.0×10¹⁴ vg/kg to about 4.0×10¹⁴ vg/kg, or 1.0×10¹⁴ vg/kg to about 1.0×10¹⁵ vg/kg, or 1.0×10¹⁴ vg/kg to about 3.0×10¹⁴ vg/kg, or about 1.0×10¹⁴ vg/kg to about 2.5×10¹⁴ vg/kg, or 1.0×10¹⁴ vg/kg to about 2.0×10¹⁴ vg/kg, or about 1.25×10¹⁴ vg/kg to about 3.75×10¹⁴ vg/kg, or about 1.25×10¹⁴ vg/kg to 6.0×10¹⁴, or about 1.25×10¹⁴ vg/kg to 5.0×10¹⁴, or about 1.25×10¹⁴ vg/kg to 4.0×10¹⁴, or about 1.25×10¹⁴ vg/kg to 1.0×10¹⁵, or about 1.25×10¹⁴ vg/kg to about 3.5×10¹⁴ vg/kg, or about 1.25×10¹⁴ vg/kg to about 3.0×10¹⁴ vg/kg, or about 1.25×10¹⁴ vg/kg to about 2.75×10¹⁴ vg/kg, or about 1.25×10¹⁴ vg/kg to about 2.5×10¹⁴ vg/kg, or about 1.25×10¹⁴ vg/kg to about 2.0×10¹⁴ vg/kg, or 1.25×10¹⁴ vg/kg to about 3.75×10¹⁴ vg/kg, or about 1.25×10¹⁴ vg/kg to about 3.5×10¹⁴ vg/kg, or 1.5×10¹⁴ vg/kg to about 1.0×10¹⁵ vg/kg, or about 1.5×10¹⁴ vg/kg to 6.0×10¹⁴, or about 1.5×10¹⁴ vg/kg to 5.0×10¹⁴, or about 1.5×10¹⁴ vg/kg to 4.0×10¹⁴, or about 1.5×10¹⁴ vg/kg to about 3.75×10¹⁴ vg/kg, or about 1.5×10¹⁴ vg/kg to about 3.5×10¹⁴ vg/kg, or about 1.5×10¹⁴ vg/kg to about 3.25×10¹⁴ vg/kg, or about 1.5×10¹⁴ vg/kg to about 3.0×10¹⁴ vg/kg, or about 1.5×10¹⁴ vg/kg to about 2.75×10¹⁴ vg/kg, or about 1.5×10¹⁴ vg/kg to about 2.5×10¹⁴ vg/kg, or about 1.5×10¹⁴ vg/kg to about 2.0×10¹⁴ vg/kg, or 1.75×10¹⁴ vg/kg to about 1.0×10¹⁵ vg/kg, or about 1.75×10¹⁴ vg/kg to 6.0×10¹⁴, or about 1.75×10¹⁴ vg/kg to 5.0×10¹⁴, or about 1.75×10¹⁴ vg/kg to 4.0×10¹⁴, or about 1.75×10¹⁴ vg/kg to about 3.75×10¹⁴ vg/kg, or about 1.75×10¹⁴ vg/kg to about 3.5×10¹⁴ vg/kg, or about 1.75×10¹⁴ vg/kg to about 3.25×10¹⁴ vg/kg, or about 1.75×10¹⁴ vg/kg to about 3.0×10¹⁴ vg/kg, or about 1.75×10¹⁴ vg/kg to about 2.75×10¹⁴ vg/kg, or about 1.75×10¹⁴ vg/kg to about 2.5×10¹⁴ vg/kg, or about 1.75×10¹⁴ vg/kg to about 2.25×10¹⁴ vg/kg, or about 1.75×10¹⁴ vg/kg to about 2.0×10¹⁴ vg/kg, or about 2.0×10¹⁴ vg/kg to 1.0×10¹⁵, or about 2.0×10¹⁴ vg/kg to 6.0×10¹⁴, or about 2.0×10¹⁴ vg/kg to 5.0×10¹⁴, or about 2.0×10¹⁴ vg/kg to about 4.0×10¹⁴ vg/kg, or about 2.0×10¹⁴ vg/kg to about 3.75×10¹⁴ vg/kg, or about 2.0×10¹⁴ vg/kg to about 3.5×10¹⁴ vg/kg, or about 2.0×10¹⁴ vg/kg to about 3.25×10¹⁴ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In one embodiment, the medicaments of the invention are formulated for systemic administration of a dose of rAAV wherein the systemic route of administration is an intravenous route and the dose of the rAAV administered is about 2.0×10¹⁴ vg/kg. In another embodiment, the medicament of the invention is formulated for systemic administration of a dose of rAAV wherein the systemic route of administration is an intravenous route and the dose of the rAAV is about 5.0×10¹² vg/kg, or about 6.0×10¹² vg/kg, or about 7.0×10¹² vg/kg, or about 8.0×10¹² vg/kg, or about 9.0×10¹² vg/kg, or about 1.0×10¹³ vg/kg, or about 1.25×10¹³ vg/kg, or about 1.5×10¹³ vg/kg, or about 1.75×10¹³ vg/kg, or about 2.25×10¹³ vg/kg, or about 2.5×10¹³ vg/kg, or about 2.75×10¹³ vg/kg, or about 3.0×10¹³ vg/kg, or about 3.25×10¹³ vg/kg, or about 3.5×10¹³ vg/kg, or about 3.75×10¹³ vg/kg, or about 4.0×10¹³ vg/kg, or about 5.0×10¹³ vg/kg, or about 6.0×10¹³ vg/kg, or about 7.0×10¹³ vg/kg, or about 8.0×10¹³ vg/kg, or about 9.0×10¹³ vg/kg, or about 1.0×10¹⁴ vg/kg, or about 1.25×10¹⁴ vg/kg, or about 1.5×10¹⁴ vg/kg, or about 1.75×10¹⁴ vg/kg, or about 2.25×10¹⁴ vg/kg, or about 2.5×10¹⁴ vg/kg, or about 2.75×10¹⁴ vg/kg, or about 3.0×10¹⁴ vg/kg, or about 3.25×10¹⁴ vg/kg, or about 3.5×10¹⁴ vg/kg, or about 3.75×10¹⁴ vg/kg, or about 4.0×10¹⁴ vg/kg, or about 5.0×10¹⁴ vg/kg, or about 6.0×10¹⁴ vg/kg, or about 1×10¹⁵ vg/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the uses of the invention, the medicament comprises a dose of rAAV in about 5 mL/kg to about 15 mL/kg, or about 8 mL/kg to about 12 mL/kg, or 8 mL/kg to about 10 mL/kg, or 5 mL/kg to about 10 mL/kg or about 10 mL/kg to 12 mL/k, or about 10 mL/kg to 15 mL/kg or 10 mL/kg to about 20 mL/kg. In a particular embodiment, the dose or the rAAV is in about 10 mL/kg. In one embodiment, the rAAV is AAVrh74.MHCK7.microdystrophin. In one embodiment, the AAVrh74.MHCK7.microdystrophin is the AAVrh74.MHCK7.microdystrophin of SEQ ID NO: 9, nucleotides 55-5021 of SEQ ID NO: 3, nucleotides 1-4977 of SEQ ID NO: 8, or nucleotides 56-5022 of SEQ ID NO: 6. In one embodiment, the rAAV is AAVrh74.MCK.microdystrophin. In one embodiment, the AAVrh74.MCK.microdystrophin is the AAVrh74.MCK.microdystrophin of nucleotides 56-4820 of SEQ ID NO: 5.

In any of the uses of the invention, the medicament is formulated for administration by injection, infusion or implantation. For example, the medicament is formulated for administration by infusion over approximately one hour. In addition, the medicament is formulated for intravenous administration through a peripheral limb vein, such as a peripheral arm vein or a peripheral leg vein. Alternatively, the infusion may be administered over approximately 30 minutes, or approximately 1.5 hours, or approximately 2 hours, or approximately 2.5 hours or approximately 3 hours.

In any of the uses of the invention, the medicament comprises an rAAV comprising the human micro-dystrophin nucleotide sequence of SEQ ID NO: 1 and the MHCK7 promoter sequence of SEQ ID NO: 2 or SEQ ID NO:7 or the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

A particular use of the invention is for preparation of a medicament for the treatment of Duchenne muscular dystrophy or Becker's muscular dystrophy. For example, the invention provides for use of a dose of recombinant adeno-virus associated (rAAV) rAAV.MHCK7.microdystrophin for the preparation of a medicament for treating Duchenne muscular dystrophy in a or Becker's muscular dystrophy human subject in need thereof, wherein the medicament is formulated for administration by intravenous infusion over approximately one hour and the dose of the rAAV administered is about 2×10¹⁴ vg/kg, and wherein the rAAV comprises the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6.

In a further embodiment, the invention provides for use of a rAAV for preparation of a medicament for reducing fibrosis in a subject in need thereof. For example, the subject in need can be suffering from a muscular dystrophy, such as DMD or any other dystrophin associated muscular dystrophy.

In another embodiment, the invention provides for use of a rAAV for the preparation of a medicament to prevent fibrosis in a subject suffering from a muscular dystrophy.

In addition, the invention provides for use of a rAAV for the preparation of a medicament to increase muscular strength and/or muscle mass in a subject suffering from muscular dystrophy.

The invention also provides for use of the rAAV for the preparation of a medicament for treatment of muscular dystrophy.

The invention provides for use of a rAAV vector comprising the human micro-dystrophin nucleotide sequence of SEQ ID NO: 1 and the MHCK7 promoter nucleotide sequence of SEQ ID NO: 2 or SEQ ID NO:7 for preparation of a medicament for the treatment of a muscular dystrophy or a rAAV vector comprising the AAVrf74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for treatment of muscular dystrophy.

In other embodiments of any of the uses of the invention, the serum CK level in the subject is decreased after administration of the rAAV to the subject as compared to the serum CK level before administration of the rAAV by a percentage level selected from the group consisting of:
a) at least 78% by 90, 180, or 270 days after the administration;
b) at least 46, 55, 70, or 95% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration;
g) at least 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration; and
h) 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

In any of the compositions for treating a muscular dystrophy or the uses of a medicament for treating a muscular dystrophy, the level of micro-dystrophin gene expression in a cell of the subject is increased after administration of the composition or medicament. Expression of the micro-dystrophin gene in the cell is detected by measuring the micro-dystrophin protein level by Western blot in muscle biopsied before and after administration of the composition or medicament. In particular, the level of micro-dystrophin protein is increased by at least about 70% to at least about 80%, or at least about 70% to at least about 90%, or at least about 80% to at least about 90% after administration of the composition or medicament compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition compared to the level of micro-dystrophin before administration of the composition or medicament.

In addition, expression of the micro-dystrophin gene in the cell is detected by measuring the micro-dystrophin protein level by immunohistochemistry in muscle biopsies before and after administration of the composition or medicament. The level of micro-dystrophin protein is increased by at least about 70% to at least about 80%, or at least about 70% to at least about 90%, or at least about 80% to at least about 90% after administration of rAAV compared to the level of micro-dystrophin before administration of the composition or medicament. For example, the level of micro-dystrophin protein is increased by at least about 70% or at least about 71% or at least about 72% or at least about 73% or at least about 74% or at least about 75% or at least about 76% or at least about 77% or at least about 78% or at least about 79% or at least about 80%, or at least about 81%, or at least about 82%, or at least about 83%, or at least about 84%, or at least about 85% after administration of the composition or medicament compared to the level of micro-dystrophin before administration of the composition or medicament.

In any of the compositions for treating a muscular dystrophy, the serum CK level in the subject is decreased after administration of the rAAV as compared to serum CK level before administration of the composition or medicament. For example, the serum CK level in the subject is decreased by about 65% to about 90% or about 65% to about 95% or about 75% to about 90% or about 80% to about 90% or about 85% to about 95% or about 87% to about 95% or about 87% to about 90% by 60 days after administration of the composition or medicament as compared to the serum CK level before administration of the composition or medicament. In particular, in any of the compositions for treating a muscular dystrophy of the invention, the serum CK level in the subject is decreased by about 87% by 60 days after administration of the composition or medicament as compared to the serum CK level before administration of the composition or medicament or in any of the compositions for treating a muscular dystrophy or the uses of a medicament for treating a muscular dystrophy of the invention, the serum CK level in the subject is decreased by about 72% by 60 days after administration of the composition or medicament as compared to the serum CK level before administration of the composition or medicament, or in any of the compositions for treating a muscular dystrophy of the invention, the serum CK level in the subject is decreased by about 73% by 60 days after administration of the composition or medicament as compared to the serum CK level before administration of the composition or medicament, or in any of the compositions for treating a muscular dystrophy or the uses of a medicament for treating a muscular dystrophy of the invention, the serum CK level in the subject is decreased by about 78% by 60 days after administration of the composition or medicament as compared to the serum CK level before administration of the composition or in any of the compositions for treating a muscular dystrophy or the uses of a medicament for treating a muscular dystrophy of the invention, the serum CK level in the subject is decreased by about 95% by 60 days after administration of the composition or medicament as compared to the serum CK level before administration of the composition or medicament. In any of the composition s for treating a muscular dystrophy or the uses of a medicament for treating a muscular dystrophy, the number of micro-dystrophin positive fibers in the muscle tissue of the subject is increased after administration of the composition or medicament as compared to the number of micro-dystrophin positive fibers before administration of the composition or medicament. For example, the number of micro-dystrophin positive fibers is detected by measuring the micro-dystrophin protein level by Western blot or immunohistochemistry on muscle biopsies before and after administration of the composition or medicament.

In any of the compositions for treating a muscular dystrophy or the uses of a medicament for treating a muscular dystrophy, administration of the composition or medicament upregulates expression of DAPC proteins such as alpha-sarcoglycan or beta-sarcoglycan. For example, the level of alpha-sarcoglycan in the subject is increased after administration of the composition or medicament as compared to the level of alpha-sarcoglycan before administration of the composition or medicament. In addition, the level of beta-sarcoglycan in the subject is increased after administration of the composition or medicament as compared to the level of the beta-sarcoglycan before administration of the composition or medicament. The level of alpha-sarcoglycan or beta-sarcoglycan is detected by measuring the alpha-sarcoglycan or beta-sarcoglycan protein level by Western blot or immunohistochemistry on muscle biopsies before and after administration of the composition or medicament.

In any of the compositions for treating a muscular dystrophy or the uses of a medicament for treating a muscular dystrophy, disease progression in the subject is delayed after administration of the composition or medicament as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, after administration of any of the compositions for treating a muscular dystrophy or the uses of a medicament for treating a muscular dystrophy, the subject has at least a 6-point improvement in NSAA score at least 270 days after administration of the composition or medicament as compared to NSAA score before administration of the rAAV. Further, in any of the methods, the subject has at least about 0.8 second improvement in time to rise at least 270 days after administration of the composition or medicament as compared to time to rise before administration of the composition or medicament. In addition, in any of the methods or uses of the invention, the subject has at least about 1.2 second improvement in time to ascend 4 steps test at least 270 days after administration of the composition or medicament as compared to time to ascend 4 steps test before administration of the composition or medicament. In addition, in any of the methods or uses of the invention, the subject has at least about 7 second improvement in the 100 m timed test at least 270 days after administration of the composition or medicament as compared to the 100 m timed test before administration of the composition or medicament.

In another embodiment, the invention provides for compositions for expressing micro-dystrophin gene in a patient cell comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6. In a further embodiment, the invention provides for use of a dose of a AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8 or nucleotides 56-5022 of SEQ ID NO: 6 for the preparation of a medicament for expressing micro-dystrophin gene in a patient cell. For example, expression of the micro-dystrophin gene in the patient cell is detected by measuring the micro-dystrophin protein level by Western blot or immunohistochemistry in muscle biopsies before and after administration of the rAAV.MHCK7.micro-dystrophin construct. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In a further embodiment, the invention provides for compositions for decreasing serum CK levels in a patient in need thereof, the composition comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the invention provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for decreasing serum CK levels in a patient in need thereof. For example, the serum CK level in the patient is decreased by at least about 65% to about 90% or about 65% to about 95% or about 75% to about 90% or about 80% to about 90% or about 85% to about 95% or about 87% to about 95% or about 87% to about 90% by 60 days after administration of the composition or medicament as compared to the serum CK level before administration of the composition or medicament. In particular, the serum CK level in the subject is decreased by about 87% by 60 days after administration of the composition or medicament as compared to the serum CK level before administration of the composition or medicament, or decreased by about 72% by 60 days after administration of the composition or medicament as compared to the serum CK level before administration of the composition or medicament, or decreased by about 73% by 60 days after administration of the composition or medicament as compared to the serum CK level before administration of the composition or medicament, or decreased by about 78% by 60 days after administration of the composition or medicament as compared to the serum CK level before administration of the composition or medicament, or decreased by about 95% by 60 days after administration of the composition or medicament as compared to the serum CK level before administration of the composition or medicament.

The invention also provides for compositions for increasing micro-dystrophin positive fibers in a patient muscle tissue comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. In addition, the invention provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing micro-dystrophin positive fibers in a patient muscle tissue. For example, the number of micro-dystrophin positive fibers is detected by measuring the dystrophin protein level by Western blot or immunohistochemistry on muscle biopsies before and after administration of the composition or medicament. In addition, the expression of the micro-dystrophin gene is measured in the patient by detecting greater the number of vector genomes per nucleus, wherein 1 vector genome per nucleus is about 50% micro-dystrophin expression and great than 1 copy per nucleus is consistent with micro-dystrophin expression level. For example, the cells have 1.2 vector copies per nucleus, or 1.3 vector copies per nucleus, or 1.4 vector copies per nucleus, or 1.5 vector copies per nucleus, or 1.6 vector copies per nucleus, or 1.7 vector copies per nucleus, or 1.8 vector copies per nucleus, or 1.9 vector copies per nucleus.

In another embodiment, the invention provides for compositions for increasing the expression of alpha-sarcoglycan in a patient in need thereof comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The invention also provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of alpha-sarcoglycan in a patient in need thereof. For example, the level of alpha-sarcoglycan is detected by measuring the alpha-sarcoglycan protein level by Western blot or immunohistochemistry on muscle biopsies before and after administration of the composition or medicament.

In addition, the invention provides for compositions for increasing the expression of beta-sarcoglycan in a patient in need thereof comprising the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6. The invention also provides for use of the AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for increasing the expression of beta-sarcoglycan in a patient in need thereof. For example, the level of beta-sarcoglycan is detected by measuring the beta-sarcoglycan protein level by Western blot or immunohistochemistry on muscle biopsies before and after administration of the composition or medicament.

The invention also provides for use of a dose of AAVrh74.MHCK7.micro-dystrophin construct nucleotide sequence of SEQ ID NO: 9, nucleotides 55-5021 of SEQ ID NO: 3, nucleotides 1-4977 of SEQ ID NO: 8, or nucleotides 56-5022 of SEQ ID NO: 6 for the preparation of a medicament for treating a patient with Duchenne muscular dystrophy or Becker muscular dystrophy, such that administration of the medicament results in disease progression in the patient is delayed as measured by any of: the six minute walk test, time to rise, ascend 4 steps, ascend and descend 4 steps, North Star Ambulatory Assessment (NSAA), 10 meter timed test, 100 meter timed test, hand held dynamometry (HHD), Timed Up and Go, and/or Gross Motor Subtest Scaled (Bayley-III) score.

For example, the subject has at least a 6-point improvement in NSAA score at least 270 days after administration of the composition or medicament as compared to NSAA score before administration of the composition or medicament. Further, the subject has at least about 0.8 second improvement in time to rise at least 270 days after administration of the composition or medicament as compared to time to rise before administration of the composition or medicament. In addition, the subject has at least about 1.2 second improvement in time to ascend 4 steps test at least 270 days after administration of the composition or medicament as compared to time to ascend 4 steps test before administration of the composition or medicament. In addition, the subject has at least about 7 second improvement in the 100 m timed test at least 270 days after administration of the composition or medicament as compared to the 100 m timed test before administration of the composition or medicament.

### BRIEF DESCRIPTION OF THE DRAWINGS

FIG. 1 illustrates the rAAV.MHCK7.micro-dystrophin construct. In this construct, the cDNA expression cassette is flanked by AAV2 inverted terminal repeat sequences (ITR). The construct is characterized by an in-frame rod deletion (R4-R23), while hinges 1, 2 and 4 (H₁, H₂ and H₄) and the cysteine rich domain remain producing a 138 kDa protein. The expression of the micro-dystrophin protein (3579 bp) is guided by a MHCK7 promoter (795 bp). The intron and 5′ UTR are derived from plasmid pCMVβ (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8, 253-261 (2002)).

FIG. 2 provides the nucleic acid sequence (SEQ ID NO: 3) AAVrh74.MHCK7.micro-dystrophin.

FIG. 3 provides the pNLREP2-Caprh74 AAV helper plasmid map.

FIG. 4 provides the Ad Helper plasmid pHELP.

FIG. 5 illustrates the rAAV.MCK.micro-dystrophin plasmid construct.

FIG. 6 provides the nucleic acid sequence (SEQ ID NO: 5) rAAVrh74.MCK.micro-dystrophin.

FIG. 7 demonstrates micro-dystrophin gene expression in muscle fibers of gastrocnemius muscle biopsy as measured by immunocytochemistry.

FIGS. 8A-8C provide Western blots demonstrating micro-dystrophin protein expression at the correct molecular weight. In FIG. 8C, Subject 4 samples (*) were diluted 1:4 (to linear range) as ULDQ (>80%) exceeded in initial analysis, and mean values were multiplied by the dilution correction factor for final value in comparison to normal. Mean Micro-dystrophin Expression Vs. Normal was 182.7% in Method 1 and 222.0% in Method 2.

FIGS. 9A-9C demonstrates administration of rAAVrh74.MHCK7. micro-dystrophin upregulates expression of the DAPC proteins, alpha-sarcoglycan and beta-sarcoglycan.

FIG. 10 demonstrates sustained dramatic reduction in Creatine Kinase (CK) with administration of rAAVrh74.MHCK7. micro-dystrophin.

FIG. 11 provides the mean CK change from baseline to day 270. This data demonstrated that CK significantly decreases over time after administration of rAAVrh74.MHCK7.micro-dystrophin.

FIG. 12 provides the mean NSAA change and the mean CK change from baseline to day 270. This data demonstrated that NSAA significantly increased over time after administration of rAAVrh74.MHCK7.micro-dystrophin.

FIG. 13 provides the nucleic acid sequence (SEQ ID NO: 9) AAVrh74.MHCK7.micro-dystrophin.

FIG. 14 illustrates the AAVrh74.MHCK7.micro-dystrophin plasmid construct.

FIG. 15 provides the nucleic acid sequence (SEQ ID NO: 9) of the AAVrh74.MHCK7.micro-dystrophin plasmid construct, which comprises kanamycin resistance gene.

### DETAILED DESCRIPTION

The present invention provides for gene therapy vectors, e.g. rAAV vectors, overexpressing human micro-dystrophin and methods of reducing and preventing fibrosis in muscular dystrophy patients. Muscle biopsies taken at the earliest age of diagnosis of DMD reveal prominent connective tissue proliferation. Muscle fibrosis is deleterious in multiple ways. It reduces normal transit of endomysial nutrients through connective tissue barriers, reduces the blood flow and deprives muscle of vascular-derived nutritional constituents, and functionally contributes to early loss of ambulation through limb contractures. Over time, treatment challenges multiply as a result of marked fibrosis in muscle. This can be observed in muscle biopsies comparing connective tissue proliferation at successive time points. The process continues to exacerbate leading to loss of ambulation and accelerating out of control, especially in wheelchair-dependent patients.

Without early treatment including a parallel approach to reduce fibrosis it is unlikely that the benefits of exon skipping, stop-codon read-through, or gene replacement therapies can ever be fully achieved. Even small molecules or protein replacement strategies are likely to fail without an approach to reduce muscle fibrosis. Previous work in aged mdx mice with existing fibrosis treated with AAV.micro-dystrophin demonstrated that we could not achieve full functional restoration (Liu, M., et al., Mol Ther 11, 245-256 (2005)). It is also known that progression of DMD cardiomyopathy is accompanied by scarring and fibrosis in the ventricular wall.

As used herein, the term “AAV” is a standard abbreviation for adeno-associated virus. Adeno-associated virus is a single-stranded DNA parvovirus that grows only in cells in which certain functions are provided by a co-infecting helper virus. There are currently thirteen serotypes of AAV that have been characterized. General information and reviews of AAV can be found in, for example, Carter, 1989, Handbook of Parvoviruses, Vol. 1, pp. 169-228, and Berns, 1990, Virology, pp. 1743-1764, Raven Press, (New York). However, it is fully expected that these same principles will be applicable to additional AAV serotypes since it is well known that the various serotypes are quite closely related, both structurally and functionally, even at the genetic level. (See, for example, Blacklowe, 1988, pp. 165-174 of Parvoviruses and Human Disease, J. R. Pattison, ed.; and Rose, Comprehensive Virology 3:1-61 (1974)). For example, all AAV serotypes apparently exhibit very similar replication properties mediated by homologous rep genes; and all bear three related capsid proteins such as those expressed in AAV2. The degree of relatedness is further suggested by heteroduplex analysis which reveals extensive cross-hybridization between serotypes along the length of the genome; and the presence of analogous self-annealing segments at the termini that correspond to “inverted terminal repeat sequences” (ITRs). The similar infectivity patterns also suggest that the replication functions in each serotype are under similar regulatory control.

An “AAV vector” as used herein refers to a vector comprising one or more polynucleotides of interest (or transgenes) that are flanked by AAV terminal repeat sequences (ITRs). Such AAV vectors can be replicated and packaged into infectious viral particles when present in a host cell that has been transfected with a vector encoding and expressing rep and cap gene products.

An “AAV virion” or “AAV viral particle” or “AAV vector particle” refers to a viral particle composed of at least one AAV capsid protein and an encapsidated polynucleotide AAV vector. If the particle comprises a heterologous polynucleotide (i.e. a polynucleotide other than a wild-type AAV genome such as a transgene to be delivered to a mammalian cell), it is typically referred to as an “AAV vector particle” or simply an “AAV vector”. Thus, production of AAV vector particle necessarily includes production of AAV vector, as such a vector is contained within an AAV vector particle.

### AAV

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the nucleotide sequence of the AAV serotype 2 (AAV2) genome is presented in Srivastava et al., J Virol, 45:555-564 (1983) as corrected by Ruffing et al., J Gen Virol, 75:3385-3392 (1994). As other examples, the complete genome of AAV-1 is provided in GenBank Accession No. NC_002077; the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78:6381-6388 (2004); the AAV-10 genome is provided in Mol. Ther., 13 (1): 67-76 (2006); and the AAV-11 genome is provided in Virology, 330 (2): 375-383 (2004). Cloning of the AAVrh.74 serotype is described in Rodino-Klapac., et al. Journal of translational medicine 5, 45 (2007). Cis-acting sequences directing viral DNA replication (rep), encapsidation/packaging and host cell chromosome integration are contained within the ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (e.g., at AAV2 nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158:97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA such as a gene cassette containing a promoter, a DNA of interest and a polyadenylation signal. The rep and cap proteins may be provided in trans. Another significant feature of AAV is that it is an extremely stable and hearty virus. It easily withstands the conditions used to inactivate adenovirus (56° C. to 65° C. for several hours), making cold preservation of AAV less critical. AAV may even be lyophilized. Finally, AAV-infected cells are not resistant to superinfection.

Multiple studies have demonstrated long-term (>1.5 years) recombinant AAV-mediated protein expression in muscle. See, Clark et al., Hum Gene Ther, 8:659-669 (1997); Kessler et al., Proc Nat. Acad Sc. USA, 93:14082-14087 (1996); and Xiao et al., J Virol, 70:8098-8108 (1996). See also, Chao et al., Mol Ther, 2:619-623 (2000) and Chao et al., Mol Ther, 4:217-222 (2001). Moreover, because muscle is highly vascularized, recombinant AAV transduction has resulted in the appearance of transgene products in the systemic circulation following intramuscular injection as described in Herzog et al., Proc Natl Acad Sci USA, 94:5804-5809 (1997) and Murphy et al., Proc Natl Acad Sci USA, 94:13921-13926 (1997). Moreover, Lewis et al., J Virol, 76:8769-8775 (2002) demonstrated that skeletal myofibers possess the necessary cellular factors for correct antibody glycosylation, folding, and secretion, indicating that muscle is capable of stable expression of secreted protein therapeutics.

Recombinant AAV genomes of the invention comprise nucleic acid molecule of the invention and one or more AAV ITRs flanking a nucleic acid molecule. AAV DNA in the rAAV genomes may be from any AAV serotype for which a recombinant virus can be derived including, but not limited to, AAV serotypes AAVrh.74, AAV-1, AAV-2, AAV-3, AAV-4, AAV-5, AAV-6, AAV-7, AAV-8, AAV-9, AAV-10, AAV-11, AAV-12 and AAV-13. Production of pseudotyped rAAV is disclosed in, for example, WO 01/83692. Other types of rAAV variants, for example rAAV with capsid mutations, are also contemplated. See, for example, Marsic et al., Molecular Therapy, 22 (11): 1900-1909 (2014). As noted in the Background section above, the nucleotide sequences of the genomes of various AAV serotypes are known in the art. To promote skeletal muscle specific expression, AAV1, AAV6, AAV8 or AAVrh.74 can be used.

DNA plasmids of the invention comprise rAAV genomes of the invention. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAVrh.74, AAV-8, AAV-9, AAV-10, AAV-11, AAV-12 and AAV-13. Production of pseudotyped rAAV is disclosed in, for example, WO 01/83692 which is incorporated by reference herein in its entirety.

A method of generating a packaging cell is to create a cell line that stably expresses all the necessary components for AAV particle production. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbial, and Immunol., 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mol. Cell. Biol. 5:3251 (1985); Mclaughlin et al., J. Virol., 62:1963 (1988); and Lebkowski et al., Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al. Vaccine 13:1244-1250 (1995); Paul et al. Human Gene Therapy 4:609-615 (1993); Clark et al. Gene Therapy 3:1124-1132 (1996); U.S. Pat. Nos. 5,786,211; 5,871,982; and 6,258,595. The foregoing documents are hereby incorporated by reference in their entirety herein, with particular emphasis on those sections of the documents relating to rAAV production.

The invention thus provides packaging cells that produce infectious rAAV. In one embodiment packaging cells may be stably transformed cancer cells such as HeLa cells, 293 cells and PerC.6 cells (a cognate 293 line). In another embodiment, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

Recombinant AAV (i.e., infectious encapsidated rAAV particles) of the invention comprise a rAAV genome. In exemplary embodiments, the genomes of both rAAV lack AAV rep and cap DNA, that is, there is no AAV rep or cap DNA between the ITRs of the genomes. Examples of rAAV that may be constructed to comprise the nucleic acid molecules of the invention are set out in International Patent Application No. PCT/US2012/047999 (WO 2013/016352) incorporated by reference herein in its entirety.

In an exemplary embodiment, the recombinant AAV vector of the invention is produced by the triple transfection method (Xiao et al., J Virol 72, 2224-2232 (1998) using the AAV vector plasmids rAAV.MHCK7.micro-dystrophin, pNLRep2-Caprh74 and pHelp, rAAV contains the micro-dystrophin gene expression cassette flanked by AAV2 inverted terminal repeat sequences (ITR). It is this sequence that is encapsidated into AAVrh74 virions. The plasmid contains the micro-dystrophin sequence and the MHCK7 enhancer and core promoter elements of the muscle specific promoter to drive gene expression. The expression cassette also contains an SV40 intron (SD/SA) to promote high-level gene expression and the bovine growth hormone polyadenylation signal is used for efficient transcription termination.

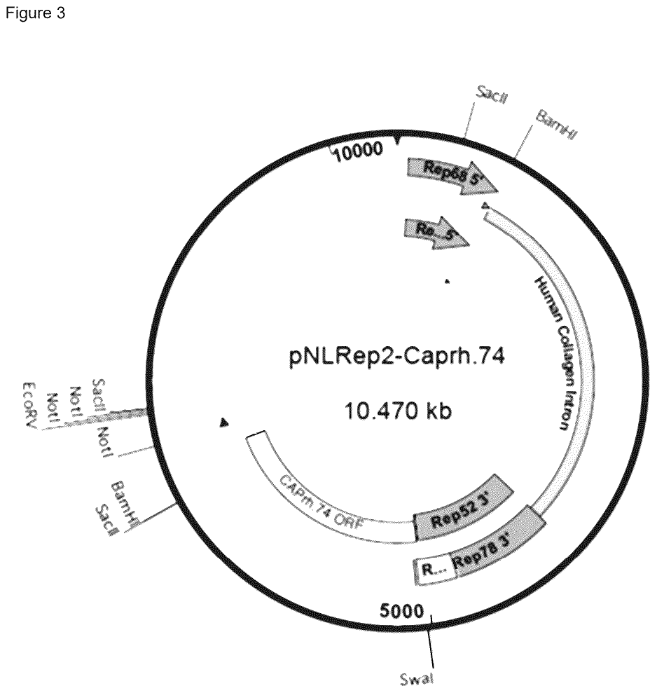

The pNLREP2-Caprh74 is an AAV helper plasmid that encodes the 4 wild-type AAV2 rep proteins and the 3 wild-type AAV VP capsid proteins from serotype rh74. A schematic map of the pNLREP2-Caprh74 plasmid is shown in FIG. 3.

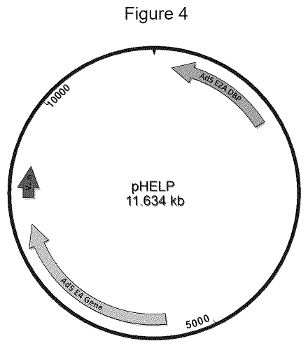

The pHELP adenovirus helper plasmid is 11,635 bp and was obtained from Applied Viromics. The plasmid contains the regions of adenovirus genome that are important for AAV replication, namely E2A, E4ORF6, and VA RNA (the adenovirus E1 functions are provided by the 293 cells). The adenovirus sequences present in this plasmid only represents ~40% of the adenovirus genome, and does not contain the cis elements critical for replication such as the adenovirus terminal repeats. Therefore, no infectious adenovirus is expected to be generated from such a production system. A schematic map of the pHELP plasmid is shown in FIG. 4.

The rAAV may be purified by methods standard in the art such as by column chromatography or cesium chloride gradients. Methods for purifying rAAV vectors from helper virus are known in the art and include methods disclosed in, for example, Clark et al., Hum. Gene Ther., 10 (6): 1031-1039 (1999); Schenpp and Clark, Methods Mol. Med., 69 427-443 (2002); U.S. Pat. No. 6,566,118 and WO 98/09657.

In another embodiment, the invention contemplates compositions comprising rAAV of the present invention. Compositions of the invention comprise rAAV and a pharmaceutically acceptable carrier. The compositions may also comprise other ingredients such as diluents and adjuvants. Acceptable carriers, diluents and adjuvants are nontoxic to recipients and are preferably inert at the dosages and concentrations employed and include buffers and surfactants such as pluronics.

Titers of rAAV to be administered in methods of the invention will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³ to about 1×10¹⁴ or more DNase resistant particles (DRP) per ml. Dosages may also be expressed in units of viral genomes (vg). One exemplary method of determining encapsilated vector genome titer uses quantitative PCR such as the methods described in (Pozsgai et al., Mol. Ther. 25 (4): 855-869, 2017).

Methods of transducing a target cell with rAAV, in vivo or in vitro, are contemplated by the invention. The in vivo methods comprise the step of administering an effective dose, or effective multiple doses, of a composition comprising a rAAV of the invention to an animal (including a human being) in need thereof. If the dose is administered prior to development of a disorder/disease, the administration is prophylactic. If the dose is administered after the development of a disorder/disease, the administration is therapeutic. In embodiments of the invention, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. An example of a disease contemplated for prevention or treatment with methods of the invention is DMD.

Combination therapies are also contemplated by the invention. Combination as used herein includes both simultaneous treatment and sequential treatments. Combinations of methods of the invention with standard medical treatments (e.g., corticosteroids) are specifically contemplated, as are combinations with novel therapies.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Route(s) of administration and serotype(s) of AAV components of the rAAV (in particular, the AAV ITRs and capsid protein) of the invention may be chosen and/or matched by those skilled in the art taking into account the infection and/or disease state being treated and the target cells/tissue(s) that are to express the micro-dystrophin protein.

The invention provides for local administration and systemic administration of an effective dose of rAAV and compositions of the invention. For example, systemic administration is administration into the circulatory system so that the entire body is affected. Systemic administration includes enteral administration such as absorption through the gastrointestinal tract and parenteral administration through injection, infusion or implantation.

In particular, actual administration of rAAV of the present invention may be accomplished by using any physical method that will transport the rAAV recombinant vector into the target tissue of an animal. Administration according to the invention includes, but is not limited to, injection into muscle and injection into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

In one embodiment of the invention, the AAVrh74.MHCK7.microdystrophin described herein is formulated in a buffer containing 20 mM Tris (pH 8.0), 1 mM magnesium chloride (MgCl₂), 200 mM sodium chloride (NaCl), and 0.001% poloxamer 188.

The dose of rAAV to be administered in methods disclosed herein will vary depending, for example, on the particular rAAV, the mode of administration, the treatment goal, the individual, and the cell type(s) being targeted, and may be determined by methods standard in the art. Titers of each rAAV administered may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 2×10¹⁴, or to about 1×10¹⁵ or more DNase resistant particles (DRP) per ml. Dosages may also be expressed in units of viral genomes (vg) (i.e., 1×10⁷ vg, 1×10⁸ vg, 1×10⁹ vg, 1×10¹⁰ vg, 1×10¹¹ vg, 1×10¹² vg, 1×10¹³ vg, 1×10¹⁴ vg, 2×10¹⁴ vg, 1×10¹⁵ vg respectively). Dosages may also be expressed in units of viral genomes (vg) per kilogram (kg) of bodyweight (i.e., 1×10¹⁰ vg/kg, 1×10¹¹ vg/kg, 1×10¹² vg/kg, 1×10¹³ vg/kg, 1×10¹⁴ vg/kg, 1.25×10¹⁴ vg/kg, 1.5×10¹⁴ vg/kg, 1.75×10¹⁴ vg/kg, 2.0×10¹⁴ vg/kg, 2.25×10¹⁴ vg/kg, 2.5×10¹⁴ vg/kg, 2.75×10¹⁴ vg/kg, 3.0×10¹⁴ vg/kg, 3.25×10¹⁴ vg/kg, 3.5×10¹⁴ vg/kg, 3.75×10¹⁴ vg/kg, 4.0×10¹⁴ vg/kg, 1×10¹⁵ vg/kg respectively). Methods for titering AAV are described in Clark et al., Hum. Gene Ther., 10:1031-1039 (1999).

In particular, actual administration of rAAV of the present invention may be accomplished by using any physical method that will transport the rAAV recombinant vector into the target tissue of an animal. Administration according to the invention includes, but is not limited to, injection into muscle and injected into the bloodstream. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as muscle. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the invention. The rAAV can be used with any pharmaceutically acceptable carrier for ease of administration and handling.

For purposes of intramuscular injection, solutions in an adjuvant such as sesame or peanut oil or in aqueous propylene glycol can be employed, as well as sterile aqueous solutions. Such aqueous solutions can be buffered, if desired, and the liquid diluent first rendered isotonic with saline or glucose. Solutions of rAAV as a free acid (DNA contains acidic phosphate groups) or a pharmacologically acceptable salt can be prepared in water suitably mixed with a surfactant such as hydroxpropylcellulose. A dispersion of rAAV can also be prepared in glycerol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques well-known to those skilled in the art.

The pharmaceutical carriers, diluents or excipients suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Transduction with rAAV may also be carried out in vitro. In one embodiment, desired target muscle cells are removed from the subject, transduced with rAAV and reintroduced into the subject. Alternatively, syngeneic or xenogeneic muscle cells can be used where those cells will not generate an inappropriate immune response in the subject.

Suitable methods for the transduction and reintroduction of transduced cells into a subject are known in the art. In one embodiment, cells can be transduced in vitro by combining rAAV with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, using e.g., a catheter.

Transduction of cells with rAAV of the invention results in sustained expression of the micro-dystrophin protein. The present invention thus provides methods of administering/delivering rAAV which express micro-dystrophin protein to an animal, preferably a human being. These methods include transducing tissues (including, but not limited to, tissues such as muscle, organs such as liver and brain, and glands such as salivary glands) with one or more rAAV of the present invention. Transduction may be carried out with gene cassettes comprising tissue specific control elements. For example, one embodiment of the invention provides methods of transducing muscle cells and muscle tissues directed by muscle specific control elements, including, but not limited to, those derived from the actin and myosin gene families, such as from the myoD gene family (See Weintraub et al., Science, 251:761-766 (1991)), the myocyte-specific enhancer binding factor MEF-2 (Cserjesi and Olson, Mol Cell Biol 11:4854-4862 (1991)), control elements derived from the human skeletal actin gene (Muscat et al., Mol Cell Biol, 7:4089-4099 (1987)), the cardiac actin gene, muscle creatine kinase sequence elements (See Johnson et al., Mol Cell Biol, 9:3393-3399 (1989)) and the murine creatine kinase enhancer (mCK) element, control elements derived from the skeletal fast-twitch troponin C gene, the slow-twitch cardiac troponin C gene and the slow-twitch troponin I gene: hypoxia-inducible nuclear factors (Semenza et al., Proc Natl Acad Sci USA, 88:5680-5684 (1991)), steroid-inducible elements and promoters including the glucocorticoid response element (GRE) (See Mader and White, Proc. Natl. Acad. Sci. USA 90:5603-5607 (1993)), and other control elements.

Muscle tissue is an attractive target for in vivo DNA delivery, because it is not a vital organ and is easy to access. The invention contemplates sustained expression of microdystrophin from transduced myofibers.

By “muscle cell” or “muscle tissue” is meant a cell or group of cells derived from muscle of any kind (for example, skeletal muscle and smooth muscle, e.g. from the digestive tract, urinary bladder, blood vessels or cardiac tissue). Such muscle cells may be differentiated or undifferentiated, such as myoblasts, myocytes, myotubes, cardiomyocytes and cardiomyoblasts.

The term “transduction” is used to refer to the administration/delivery of the coding region of the micro-dystrophin to a recipient cell either in vivo or in vitro, via a replication-deficient rAAV of the invention resulting in expression of micro-dystrophin by the recipient cell.

Thus, the invention provides methods of administering an effective dose (or doses, administered essentially simultaneously or doses given at intervals) of rAAV that encode micro-dystrophin to a subject in need thereof.

The following EXAMPLES are provided by way of illustration and not limitation. Described numerical ranges are inclusive of each integer value within each range and inclusive of the lowest and highest stated integer.

### EXAMPLES

### Example 1

### A) Generation of the AAVrh74.MHCK7.Micro-Dystrophin Construct

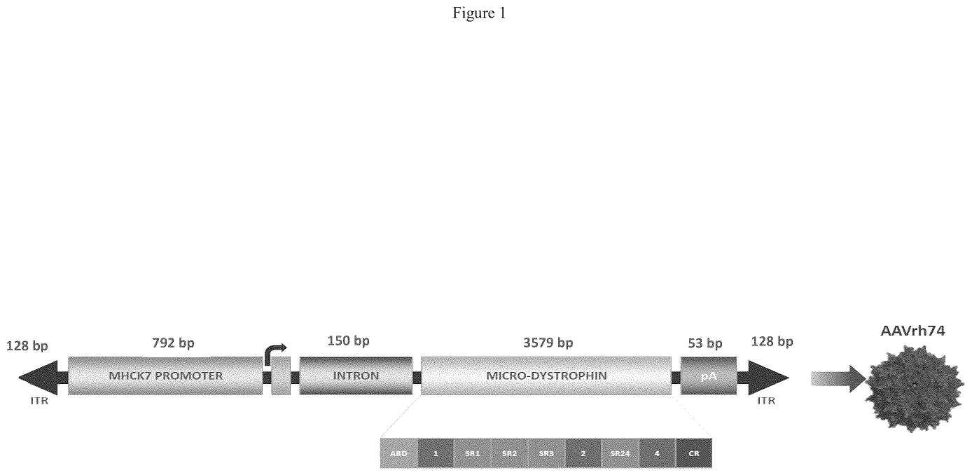

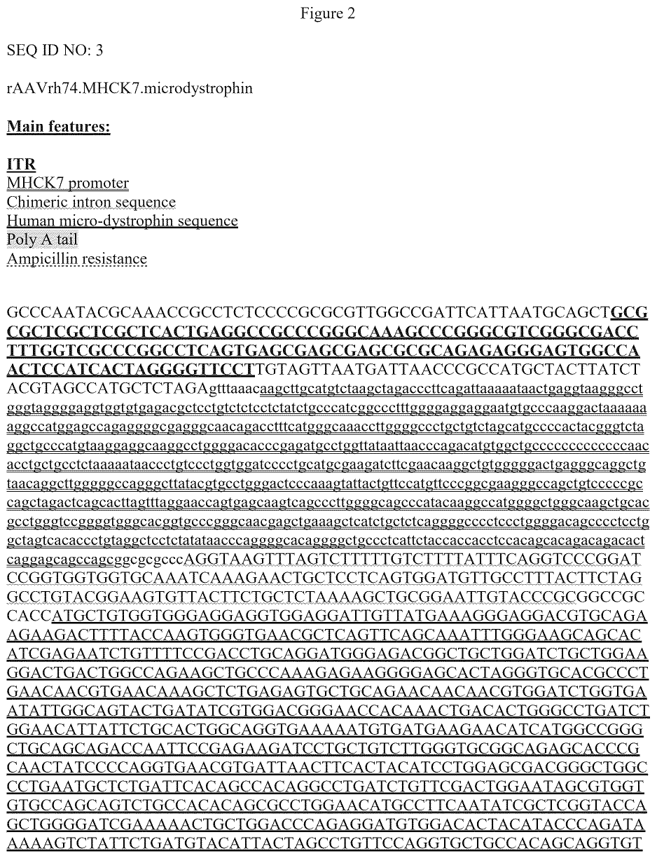

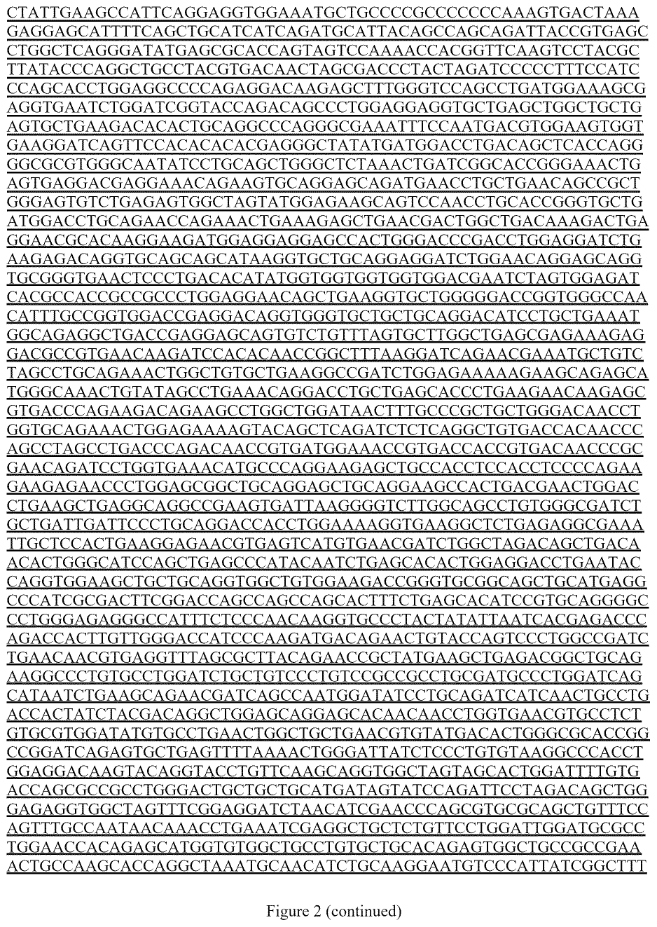

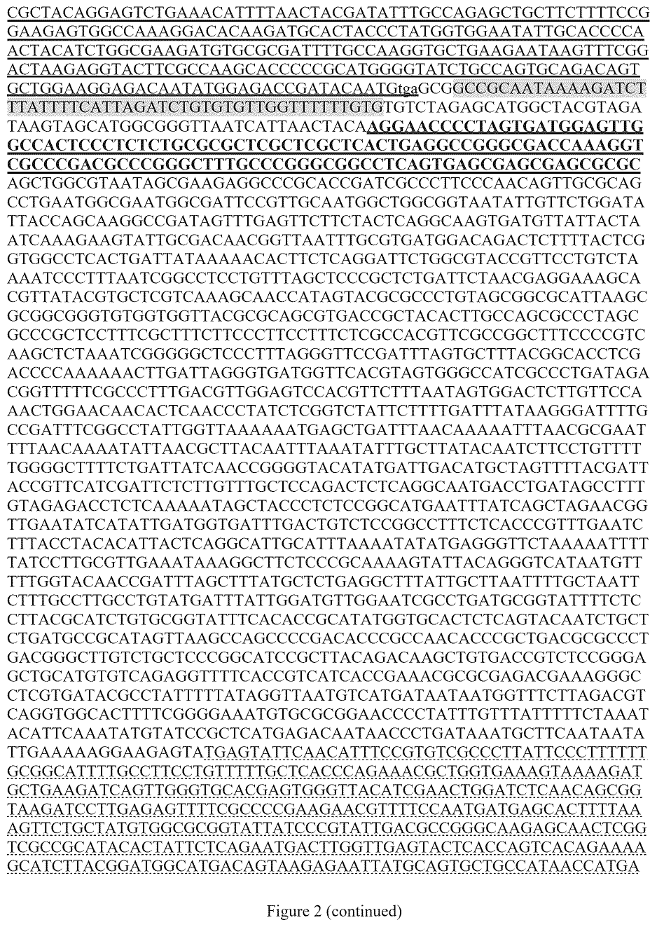

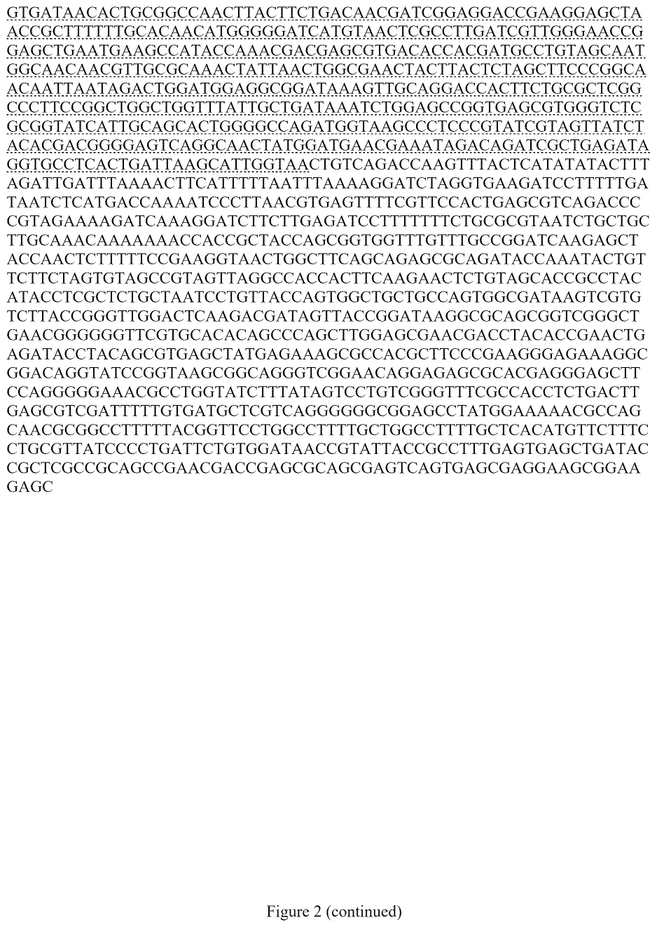

The AAVrh74.MHCK7.micro-dystrophin plasmid contains a human micro-dystrophin cDNA expression cassette flanked by AAV2 inverted terminal repeat sequences (ITR) (see FIG. 1). The micro-dystrophin construct was characterized by an in-frame rod deletion (R4-R23), while hinges 1, 2 and 4 and cysteine rich domain remain producing a 138 kDa protein. The expression of the micro-dystrophin protein (3579 bp) was guided by a MHCK7 promoter (792 bp). The plasmid was constructed from the rAAV.MCK.micro-dystrophin plasmid by removing the MCK promoter and inserting the MHCK7 promoter. After the core promoter, the 53 bp endogenous mouse MCK Exon1 (untranslated) is present for efficient transcription initiation, followed by the SV40 late 16S/19S splice signals (150 bp) and a small 5′UTR (61 bp). The intron and 5′ UTR are derived from plasmid pCMVβ (Clontech). The micro-dystrophin cassette had a consensus Kozak immediately in front of the ATG start and a small 53 bp synthetic polyA signal for mRNA termination. The human micro-dystrophin cassette contained the (R4-R23/Δ71-78) domains as previously described by Harper et al. (Nature Medicine 8, 253-261 (2002)). The complementary DNA was codon optimized for human usage and synthesized by GenScript (Piscataway, NJ) (Mol Ther 18, 109-117 (2010)). The only viral sequences included in this vector were the inverted terminal repeats of AAV2, which are required for both viral DNA replication and packaging. The micro-dystrophin cassette has a small 53 bp synthetic polyA signal for mRNA termination.

Previous studies have validated cardiac expression using MHCK7 promoter (Salva et al. Mol Ther 15, 320-329 (2007) and AAVrh74 achieving skeletal, diaphragm, and cardiac muscle expression (Sondergaard et al. Annals of clinical and Transl Neurology 2, 256-270 (2015)), The sequence of construct of FIG. 1 was encapsidated into AAVrh.74 virions. The molecular clone of the AAVrh.74 serotype was cloned from a rhesus macaque lymph node and is discussed in in Rodino-Klapac et al. Journal of Translational medicine 5, 45 (2007).

Table 1 shows the molecular features of the plasmid AAVrh74.MHCK7.micro-dystrophin (SEQ ID NO: 3)

TABLE 1
Molecular Features of plasmid rAAV•MHCK7•micro-dystrophin

| TYPE | START | END | NAME | DESCRIPTION |
|---|---|---|---|---|
| REGION | 55 | 182 | 5' ITR | Wild-type AAV2 inverted |
| | | | | terminal repeat |
| REGION | 244 | 1035 | MHCK7 | Mouse myosin heavy chain |
| | | | | complex - E box muscle |
| | | | | creatine kinase fusion |
| | | | | enhancer/promoter |
| REGION | 1045 | 1194 | Chimeric | 5' donor site from human |
| | | | intron | β-globin gene |
| | | | | and the branchpoint and 3' |
| | | | | splice acceptor site from IgG |
| | | | | heavy chain variable region |
| GENE | 1205 | 4783 | huDys | Human micro-dystrophin |
| | | | cDNA | cDNA |
| REGION | 4786 | 4838 | PolyA | Synthetic PolyA |
| REGION | 4894 | 5021 | 3' ITR | Wild-type AAV2 inverted |
| | | | | terminal repeat |
| GENE | 6760 | 7619 | AmpR | β-lactamase gene |
| REGION | 7823 | 8442 | Ori | Plasmid origin of replication |

B) Generation of the AAVrh74.MHCK7.Micro-Dystrophin Construct from and Plasmid Encoding Kanamycin (Kan) Resistance

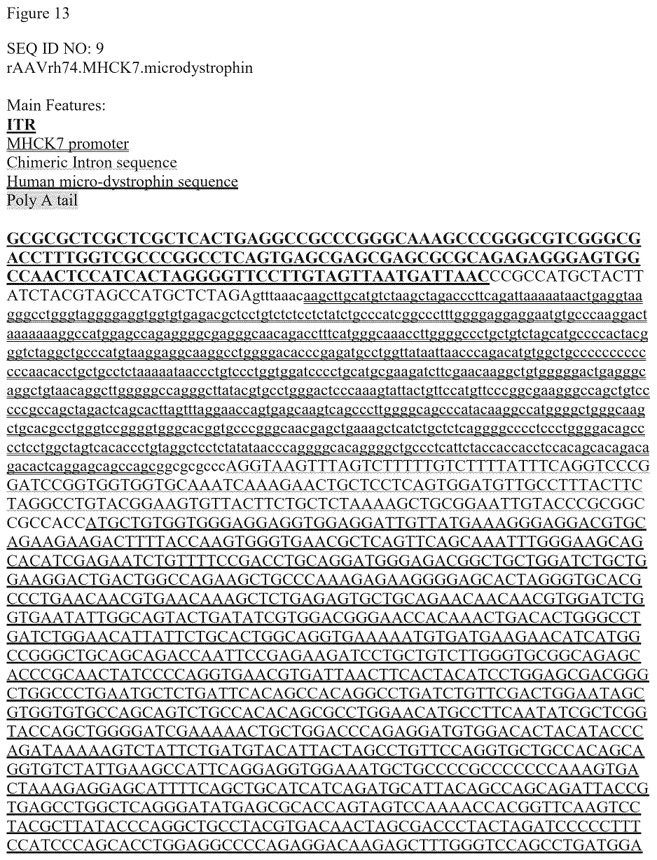

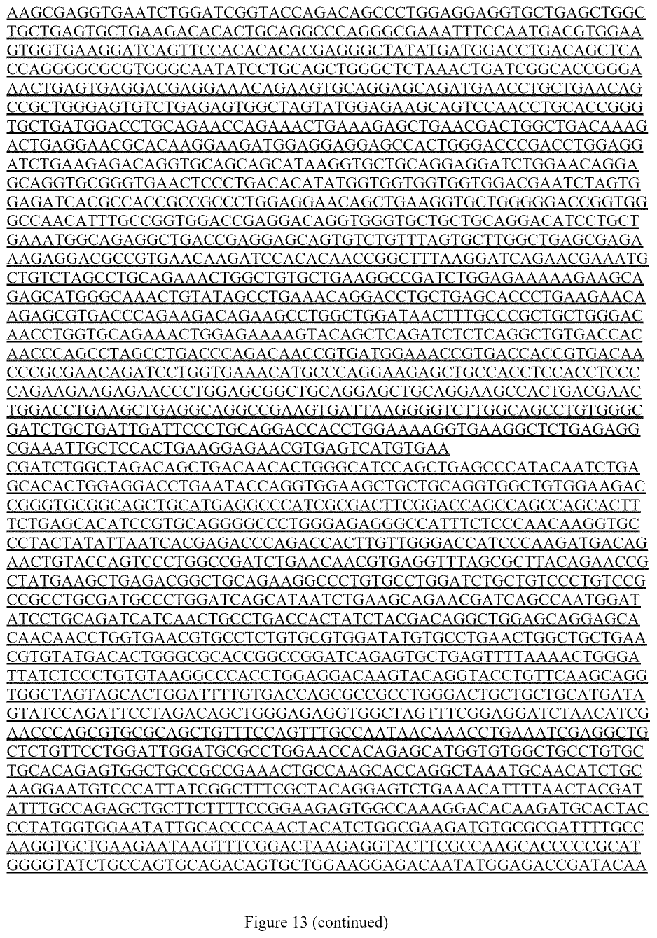

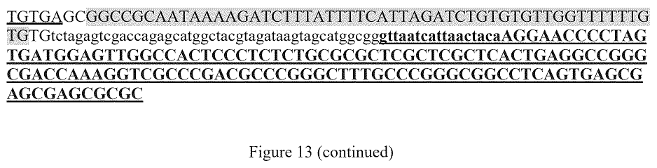

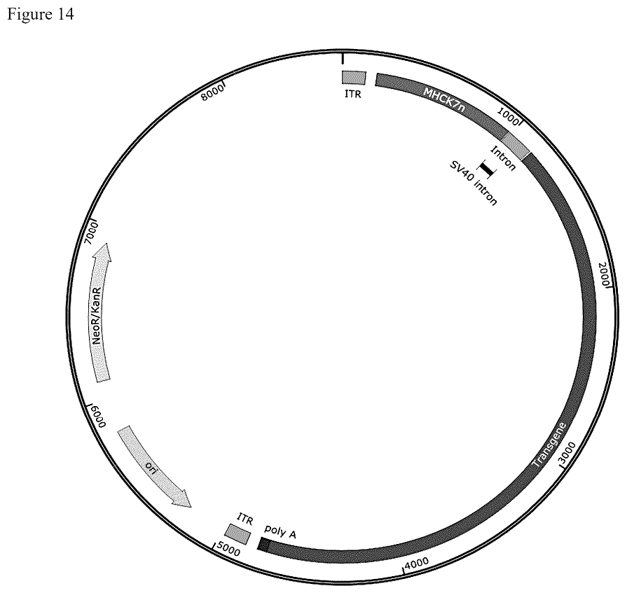

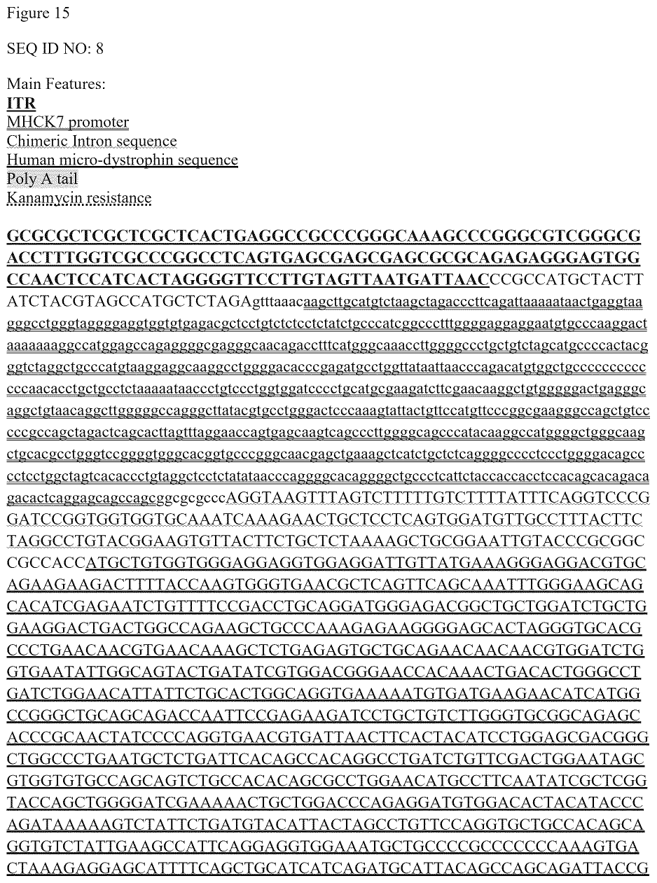

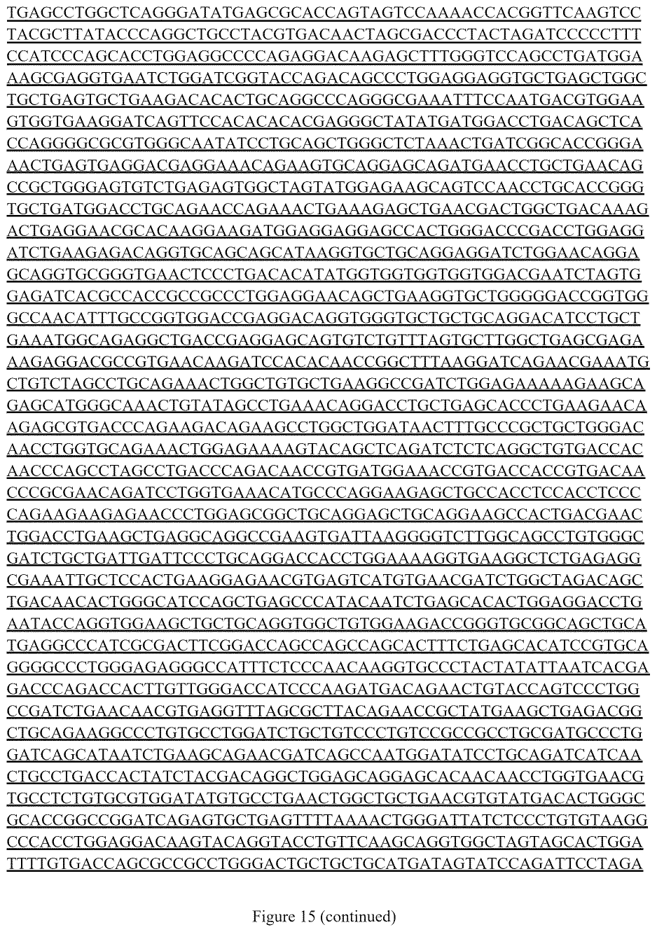

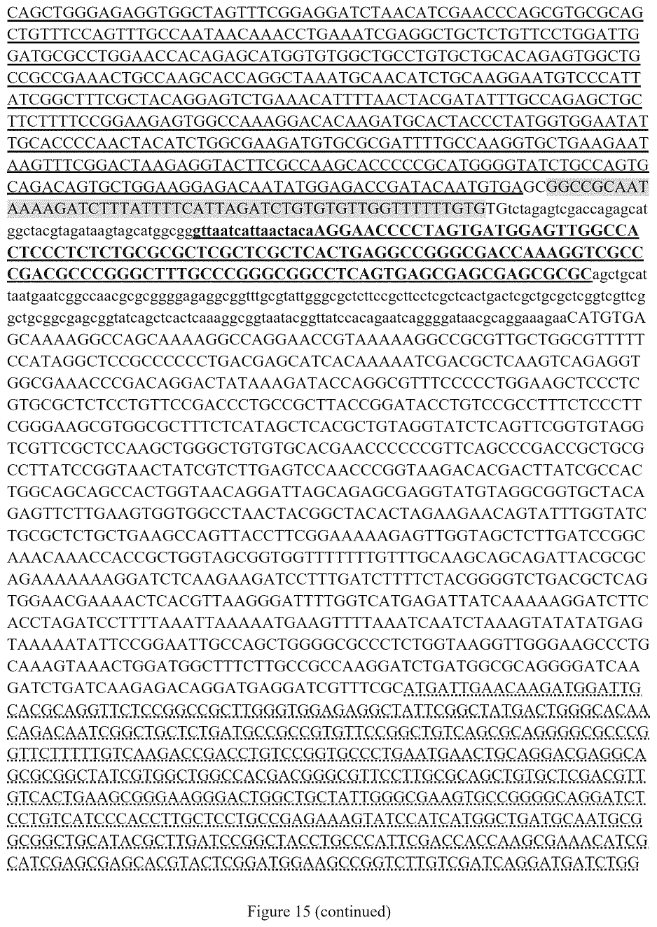

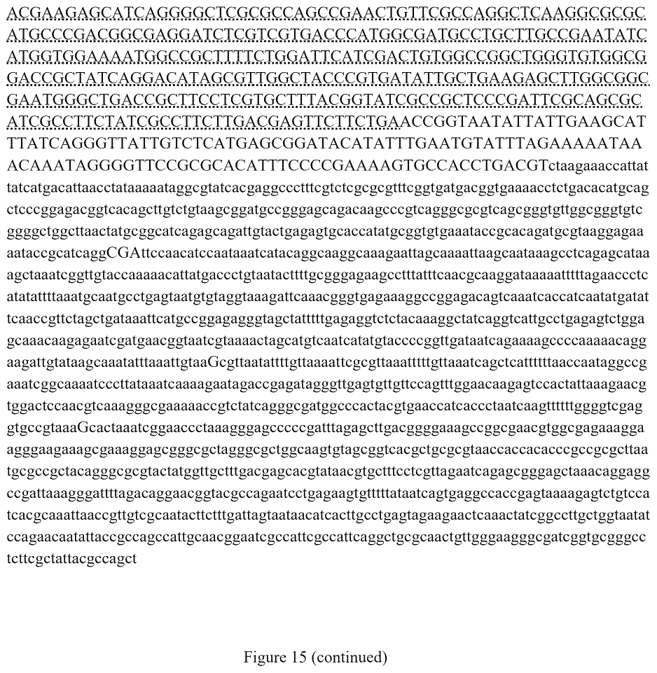

Cloning of MHCK7.μDys.KAN was achieved by isolating the MHCK7.μDys fragment from an MHCK7.μDys.AMP plasmid and the Kanamycin Backbone, and annealing them using the NEBuilder cloning workflow. The MHCK7.μDys fragment was isolated via restriction enzyme digestion with SnaBI. The digestion was performed in a 50 μL total reaction in 1× CutSmart Buffer (NEB) and 1 μL SnaBI, at 37° C. for 1 hour. The resulting fragment was isolated via electrophoresis using a 1% Agarose gel, running at 105 volts for 1.5 hours. The band corresponding to the MHCK7.μDys insert was cut out and purified using a gel purification kit (Macherey-Nagel). The resulting fragment had a DNA concentration of 10 ng/μL. The Kan backbone fragment was isolated via XbaI restriction enzyme digestion in a 50 μL reaction with 1× CutSmart Buffer (NEB) and 1 μL XbaI, at 37° C. for 1 hour. The resulting fragment was isolated via electrophoresis using a 1% Agarose gel, running at 105 volts for 1.5 hours. The band corresponding to the Kan Backbone was cut out and purified via gel purification kit (Macherey-Nagel). The resulting fragment had a DNA concentration of 8.1 ng/μL. The two fragments were annealed using the NEB Builder cloning workflow, which has the ability to join two fragments with overlapping sequences. The NEBuilder cloning reaction was performed per manufacturer protocol at 50° C. for 15 minutes, using a 1:1 ratio of MHCK7.μDys to Kanamycin backbone in 1× NEBuilder HiFi DNA Assembly Master Mix for a total reaction volume of 20 μL. The resulting clone was transformed into NEB® Stable Competent E. coli (C3040) by adding 2.5 μL cloning product to the cells followed by 30 minutes on ice, then 30 seconds at 42° C. and an additional 5 minutes on ice. After transformation, 950 μL of outgrowth media was added to the cells and allowed to grow at 30° C. for 1.5 hours, shaking at 225 rpm. Following outgrowth, 450 μL of these cells was plated on a 50 μg/mL kanamycin LB agar plate and incubated overnight at 30° C. in a dry incubator. A colony was picked from this plate and grown up overnight in LB containing 50 μg/mL kanamycin. DNA was isolated from 3 mL of this culture using QIAprep® Spin Miniprep Kit (Qiagen). This DNA was used to confirm the cloning product. The cloning product was confirmed via restriction enzyme digestion with PmeI, MscI, and SmaI followed by gel electrophoresis. The cloning product was additionally confirmed via sequencing. The resultant plasmid is set forth in SEQ ID NO:8, and shown in FIGS. 14 and 15. The sequence of construct of FIG. 13 which corresponds to that of SEQ ID NO:9, and nucleotides 1-4977 of SEQ ID NO: 8, was encapsidated into AAVrh.74 virions as described above.

### Example 2

### Systemic Gene Delivery Clinical Trial for Duchenne Muscular Dystrophy

This is a single-dose controlled trial using the rAAVrh74.MHCK7.micro-dystrophin of SEQ ID NO: 3, nucleotides 55-5021, for DMD subjects. Cohort A will include six subjects of ages 3 months to 3 years, and Cohort B will include six subjects of ages 4 years to 7 years old. All subjects will receive intravenous micro-dystrophin vector (2×10¹⁴ vg/kg in 10 mL/kg). The rAAVrh74.MHCK7.micro-dystrophin is formulated in a buffer containing 20 mM Tris (pH 8.0), 1 mM magnesium chloride (MgCl₂), 200 mM sodium chloride (NaCl), and 0.001% poloxamer 188.

In the study, the rAAVrh74.MHCK7.micro-dystrophin was infused via peripheral arm vein so that it can reach all the muscles in the body. Six DMD subjects ages 3 months to 3 years in Cohort A, and six DMD subjects ages 4 years to age 7 years in Cohort B, were enrolled. All subjects received intravenous micro-dystrophin vector (2×10¹⁴ vg/kg in 10 mL/kg). The encapsilated vector genome titer for the administered dose was determined using quantitative PCR using a Prism 7500 Taqman detector system (PE Applied Biosystems) with primers directed against the MHCK7 promoter compared to a supercoiled DNA plasmid standard (Pozsgai et al. Mol. Ther. 25 (4): 855-869, 2017).

Subjects received infusions over 1 hour in the Pediatric Intensive Care Unit (PICU) at Nationwide Children's Hospital. Before the gene therapy, a muscle biopsy was performed at the screening visit. Subjects will have a second muscle biopsy to determine if the gene allowed for replacement of the missing dystrophin protein at 90 days post-delivery. After the gene transfer, patients were carefully monitored for any side effects of the treatment. This monitoring included blood and urine tests, as well as physical examination during the screening visits and on days 0, 1, 7, 14, 30, 60, 90, and 180, and at months 9, 12, 18, 24, 30 and 36 to make sure that there are no side effects from the gene injection.

The subjects of Cohort A (n=6) were between 3 months to 3 years of age, and received intravenous rAAVrh74.MHCK7.micro-dystrophin vector (2×10¹⁴ vg/kg in 10 mL/kg). One-day prior to gene transfer subjects in Cohort A were started on prednisone or deflazacort 1 mg/kg and maintained for 30 days while monitoring immune response. If negative at day 30, steroids were weaned over 1 week. If T cell response to AAV or micro-dys was >125 SFC/106 PBMCs, steroids were maintained until levels drop below this threshold.

The subjects of Cohort B (n=6) were between 4 years to 7 years of age, and received intravenous rAAVrh74.MHCK7.micro-dystrophin vector (2×10¹⁴ vg/kg in 10 mL/kg). These subjects were maintained on stable dose of corticosteroids throughout trial but may have been increased for short time if T cell response to AAV or micro-dystrophin was >125 SFC/106 PBMCs.

### Eligibility Criteria

Inclusion criteria for the study were as follows:
Age of enrollment: Cohort A: 3 months to 7 years of age and Cohort B: between 4-7 years of age, inclusive.
Molecular characterization of the DMD gene with frameshift (deletion or duplication), or premature stop codon mutation between exons 18 to 58.
CK elevation>1000 U/L
Cohort A subjects: below average on the Bayley-III motor assessment for gross motor defined as a scaled score of ≤9
Cohort B: Below average on 100 meter timed test defined as <80% predicted
Males of any ethnic group.
Ability to cooperate with motor assessment testing.
Cohort A subjects: No previous treatment with corticosteroids.
Cohort B subjects: Stable dose equivalent of oral corticosteroids for at least 12 weeks prior to screening and the dose is expected to remain constant (except for modifications to accommodate changes in weight) throughout the stud

Exclusion criteria for the study were as follows:
Active viral infection based on clinical observations.
Signs of cardiomyopathy, including echocardiogram with ejection fraction below 40%.
Serological evidence of HIV infection, or Hepatitis B or C infection.
Diagnosis of (or ongoing treatment for) an autoimmune disease.
Abnormal laboratory values considered clinically significant
Concomitant illness or requirement for chronic drug treatment that in the opinion of the PI creates unnecessary risks for gene transfer.
Subjects with AAVrh74 or AAV8 antibody titers>1:400 as determined by ELISA immunoassay.
Medical condition or extenuating circumstance that, in the opinion of the investigator, might compromise the subject's ability to comply with the protocol required testing or procedures or compromise the subject's wellbeing, safety, or clinical interpretability.
Severe infection (e.g., pneumonia, pyelonephritis, or meningitis) within 4 weeks before gene transfer visit (enrollment may be postponed).
Received any investigational medication (other than corticosteroids) or exon skipping medications (including ExonDys 51®), experimental or otherwise, in the last 6 months prior to screening for this study.
Has had any type of gene therapy, cell based therapy (e.g. stem cell transplantation), or CRISPR/Cas9 therapy.
Family does not want to disclose patient's study participation with primary care physician and other medical providers.

### Outcome Measures

The primary outcome measure was safety based on number of participants with adverse events (time frame: 3 years). Adverse effects were monitored and scored for severity and relatedness to the study article.

The secondary outcome measures were as follows:

Gross Motor Subtest Scaled (Bayley-III) score (time frame: screening, Day 30-3 Years): The Gross Motor Scaled Score measured motor development. The Bayley-III Gross Motor Subtest was scored for Cohort A on every follow up visit starting at Day 30 through 3 years. Any subject that was 43-47 months of age, inclusive, at time of screening had the scaled score calculated compared to normative data for 42 month old children. The Bayley-III provided normative data for children 1-42 months of age.

Physical Therapy Assessments The 100 Meter Timed Test (100 m) (time frame: screening, Day 30-3 Years): The 100 m was the primary motor outcome for Cohort B. The 100 Meter Timed Test was an exploratory outcome initiated for Cohort A as soon as the child was 3 years of age.

Physical Therapy Assessments North Star Ambulatory Assessment (NSAA) (time frame: screening, Day 30-3 Years): The North Star Ambulatory Assessment (NSAA) was an exploratory outcome initiated for Cohort A as soon as the child was four years of age and for cohort B. The NSAA measures the quality of ambulation in young boys with Duchenne Muscular Dystrophy.

Physical Therapy Assessments Timed Up and Go modified for children (TUG) (time frame: screening, Day 30-3 Years): Exploratory outcomes for Cohort B included the Timed Up and Go modified for children (TUG).

Physical Therapy Assessments Ascend and Descend of 4 steps (time frame: screening, Day 30-3 Years): Exploratory outcomes for Cohort B will include ascend and descend of 4 steps.

Physical Therapy Assessments Hand Held Dynamometry (HHD) (time frame: screening, Day 30-3 Years): Exploratory outcomes for Cohort B included hand held dynamometry (HHD) for knee extensors and knee flexors, and elbow flexors and elbow extensors.

Micro-dystrophin gene expression quantification by immunofluorescence (time frame: screening, Day 90): Micro-dystrophin gene expression levels were quantified by immunofluorescence and compared in pre and post muscle biopsies.

Micro-dystrophin gene expression quantification by western blot (time frame: screening, Day 90): Micro-dystrophin gene expression levels were quantified by western blot analysis and compared in pre and post muscle biopsies.

A decrease in CK following gene therapy (time frame: 3 years): Decrease in CK levels in circulating blood.

Cardiac magnetic resonance imaging (at 1 year).

### Micro-Dystrophin Gene Expression

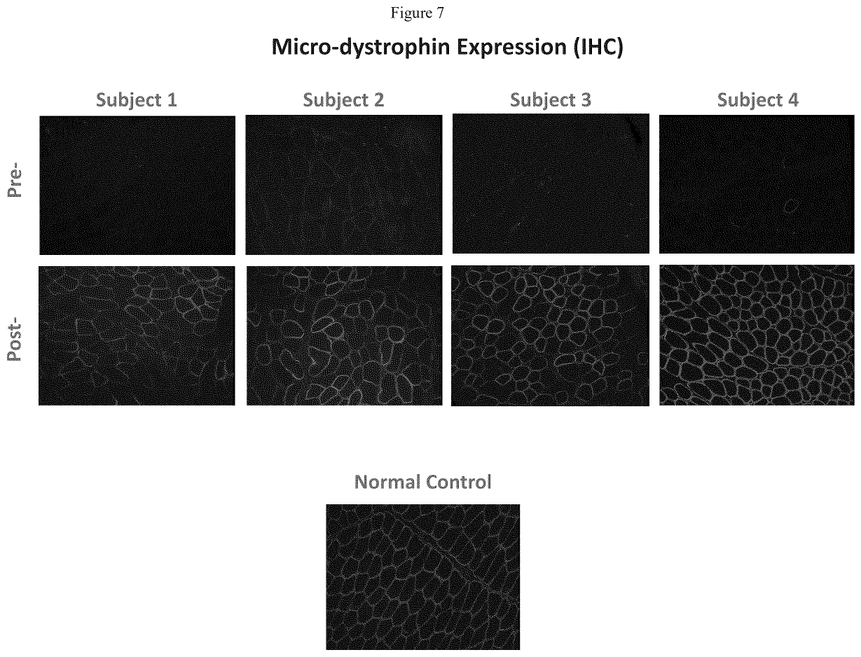

Change from Baseline in micro-dystrophin expression via immunofluorescence (IF) fiber intensity was analyzed and quantitated. As shown in FIG. 7, Subject 1 (age 5) demonstrated 78% expression, of micro-dystrophin protein in the muscle fibers of the gastrocnemius muscle biopsy after administration of the rAAVrh74.MHCK7.micro-dystrophin, Subject 2 (age 4) demonstrated 73.5% expression of micro-dystrophin protein in the muscle fibers of the gastrocnemius muscle biopsy after administration of the rAAVrh74.MHCK7.micro-dystrophin, and Subject 3 (age 6) demonstrated 77.0% expression of micro-dystrophin protein in the muscle fibers of the gastrocnemius muscle biopsy after administration of the rAAVrh74.MHCK7.micro-dystrophin. Subject 4 (age 4) demonstrated 96.2% expression of micro-dystrophin in the muscle fibers of the gastrocnemius muscle biopsy after administration of the rAAVrh74.MHCK7.micro-dystrophin. All patients showed robust expression of transduced micro-dystrophin, which is properly localized to the muscle sarcolemma, as measured by immunohistochemistry. Mean gene expression, as measured by percentage of micro-dystrophin positive fibers was 76.2% and the mean intensity of the fibers was 74.5% compared to normal control.

| | | Percentage of |
|---|---|---|
| | Mean | Dystrophin- |
| Subject | Intensity | Positive Fibers |
| 1 | 82.0% | 78.0% |
| 2 | 59.0% | 73.5% |
| 3 | 83.0% | 77.0% |
| 4 | 160.0% | 96.2% |
| Mean | 96.0% | 81.2% |

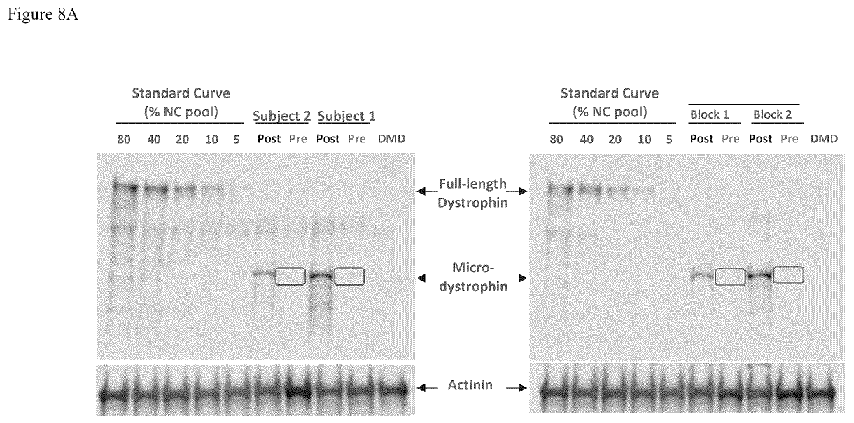

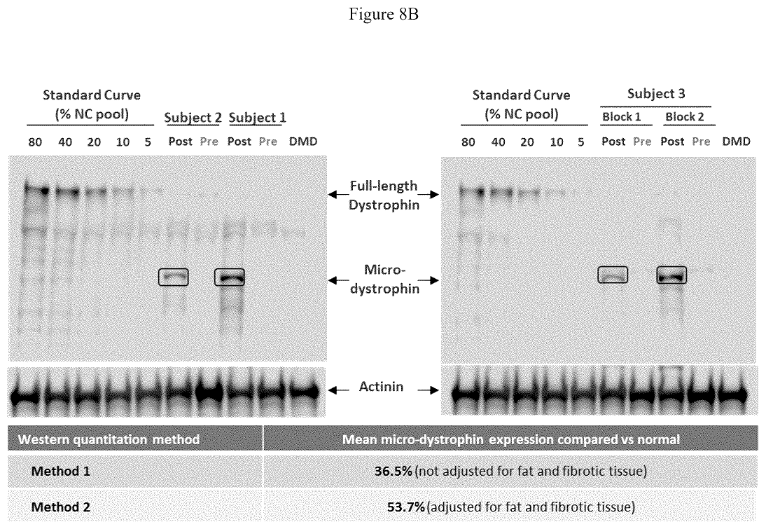

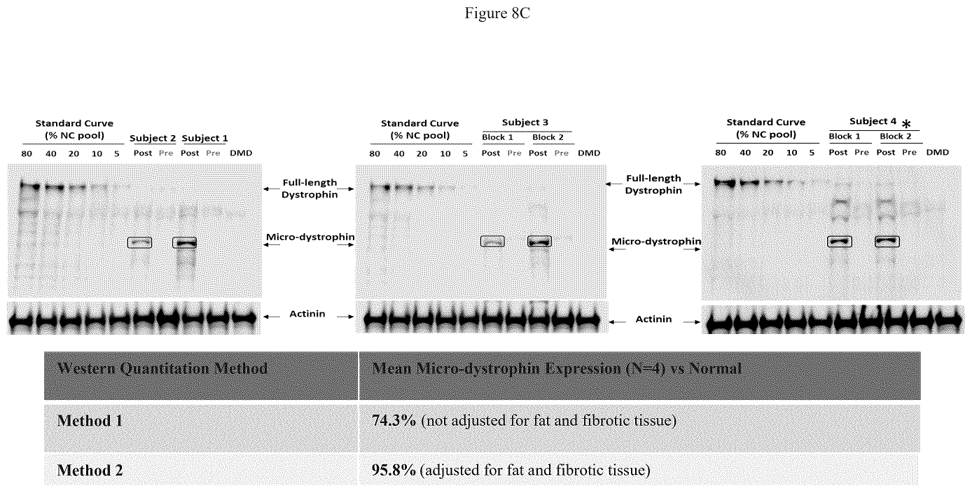

The change in micro-dystrophin gene expression from Baseline to Day 60 was also assessed by quantitating micro-dystrophin protein expression as measured by Western blot of biopsied muscle tissue. As shown in FIGS. 8A and 8B, Western blot analysis detected micro-dystrophin protein expression in Subject 1 (age 5), Subject 2 (age 4) and Subject 3 (age 6). FIG. 8C provides the Western Blot analysis detecting micro-dystrophin protein expression in Subject 4 (age 4). All post-treatment biopsies showed robust levels of micro-dystrophin as measured by Western blot, with a mean for Subjects 1-4 of 74.3 compared to normal utilizing method 1, and 95.8% compared to normal pursuant to method 2 that adjusts for fat and fibrotic tissue.

For each subject, the vector genome copy per nucleus of the muscle fibers were measured. As shown in Table 2, the vector genome copy per nuclease was greater than 1 for each of the subjects after administration of rAAVrh74.MHCK7.micro-dystrophin. One copy of the vector indicates approximately 50% expression of the micro-dystrophin gene. A mean of 1.6 vector copies per cell nucleus was measured in Subjects 1-3, consistent with the high micro-dystrophin expression levels observed. When the values for Subject 4 were included, the mean vectors copies/μg DNA is >10⁵ with a mean of 3.3 vector copies per cell nucleus.

TABLE 2

| | Vector copies/ | Copies |
|---|---|---|
| Subject | μg DNA | per Nuclei |
| 1 | >10⁵ | 1.7 |
| 2 | >10⁵ | 1.3 |
| 3 | >10⁵ | 1.9 |

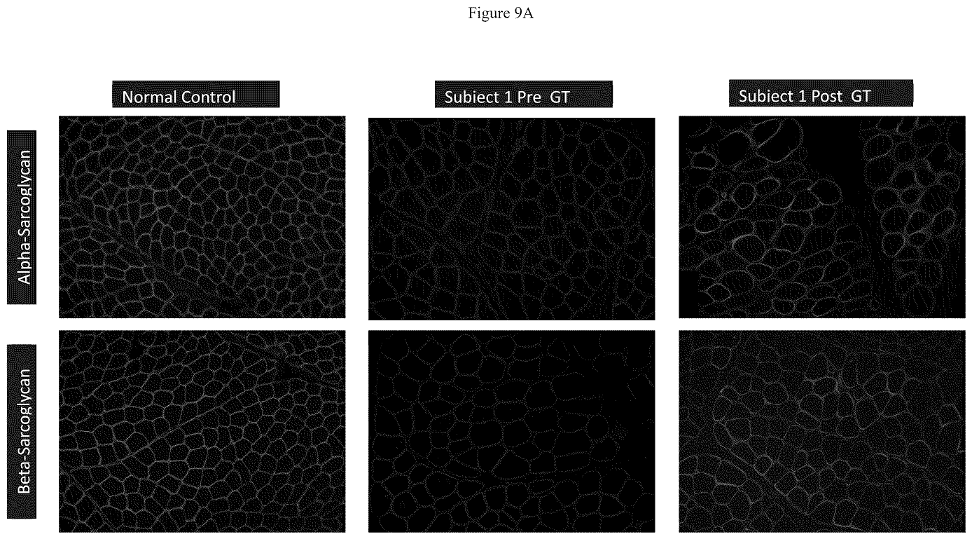

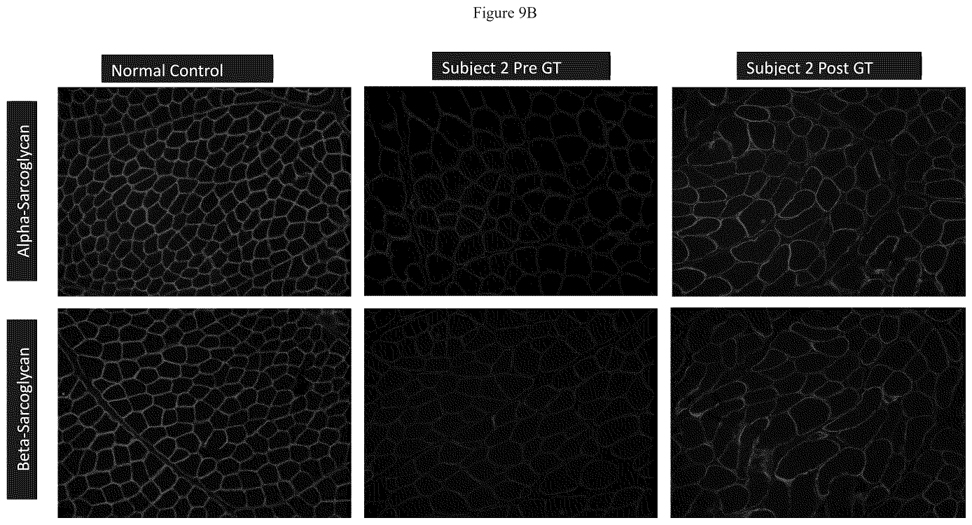

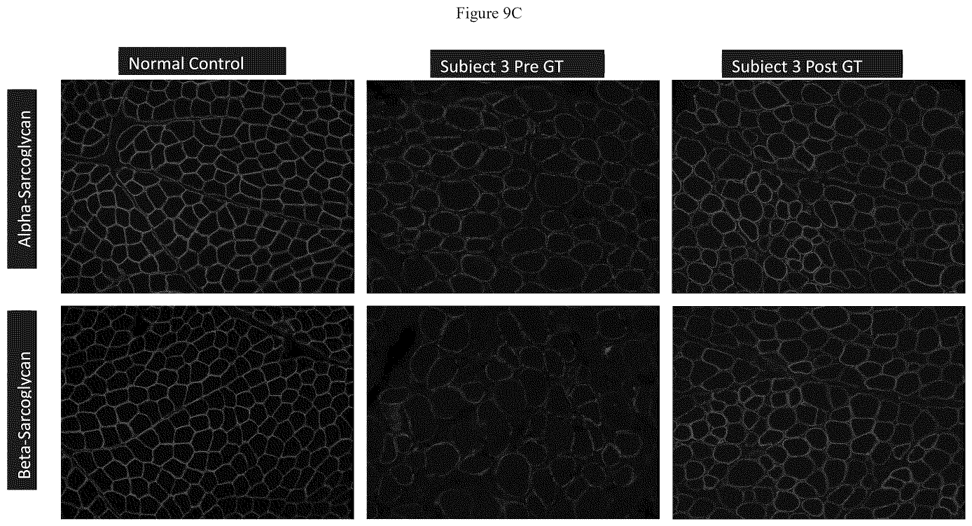

The protein levels of alpha-sarcoglycan and beta-sarcoglycan in muscle biopsy tissue were measured by immunohistochemistry before and after administration of rAAVrh74.MHCK7.micro-dystrophin. Administration of rAAVrh74.MHCK7 also resulted in upregulation of the DAPC proteins in the subjects. As shown in FIG. 9, expression of alpha-sarcoglycan and beta-sarcoglycan in muscle biopsy tissue was increased compared to the level so these proteins in muscle biopsies before administration of rAAVrh74.MHCK7 in Subjects 1 (FIG. 9A), Subject 2 (FIG. 9B) and Subject 3 (FIG. 9C).

### Circulating Serum CK Levels

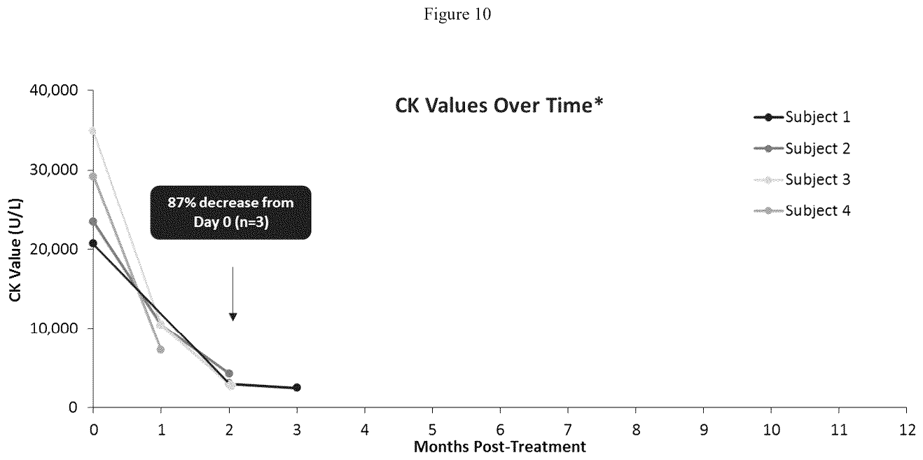

Blood samples were obtained every 30 days after administration intravenous infusion of rAAVrh74.MHCK7.micro-dystrophin vector (2×10¹⁴ vg/kg in 10 mL/kg). CK levels were measured at each visit and compared to the baseline level obtained before administration of the rAAVrh74.MHCK7.micro-dystrophin (Visit Day 0). Baseline serum CK levels (Units/Liter) are provided in Table 3 below. As shown in FIG. 10, the level of circulating serum CK decreased about 87%, 2 months after administration of rAAVrh74.MHCK7.micro-dystrophin. All subjects showed significant decreases of serum creatine kinase (CK) levels, with a mean reduction of CK of over 87% at 2 months' post-treatment (n=3). CK is an enzyme associated with muscle damage and patients with DMD uniformly exhibit high levels of CK. Indeed, significantly elevated CK is often used as a preliminary diagnosis tool for DMD, which is then followed by confirmatory genetic testing.

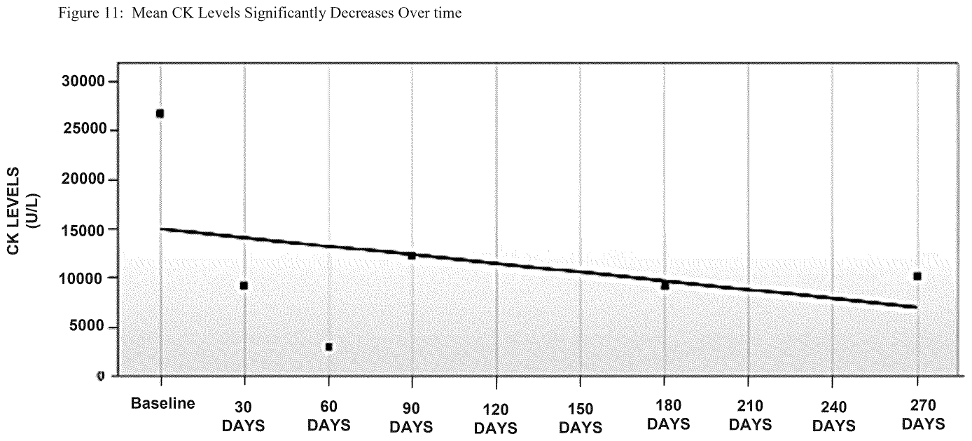

Table 4 and FIG. 10 provide the CK levels for each subject. FIG. 11 provides the mean CK levels over time, and demonstrates that the mean CK levels significantly decrease over time after administration of rAAVrh74.MHCK7.micro-dystrophin. The mean baseline CK level of 27,064 U/L (Mean for Table 3) is decreased by about 63% to a mean of 9,982 U/L (Mean, Day 270, Table 4).

TABLE 3

| | | CK Levels at |
|---|---|---|
| | Age | Baseline Units/ |
| Subject | (years) | Liter (U/L) |
| 1 | 5 | 20691 |
| 2 | 4 | 23414 |
| 3 | 6 | 34942 |
| 4 | 4 | 29210 |

TABLE 4
Change in CK levels from Baseline to Day 270

| | Base- | Day | Day | Day | Day | Day | Day |
|---|---|---|---|---|---|---|---|
| Subject | line | 30 | 60 | 90 | 180 | 270 | 360 |
| 1 | | — | 2984 | 2444 | 18476 | 6317 | — |
| 2 | 23414 | 10427 | 4283 | 41920 | 6209 | 10494 | — |
| 3 | 34942 | 10430 | 2966 | 2546 | 9650 | 18855 | 6410 |
| 4 | 29210 | 7215 | 908 | 1382 | 2580 | 4262 | — |

### Efficacy Assessment

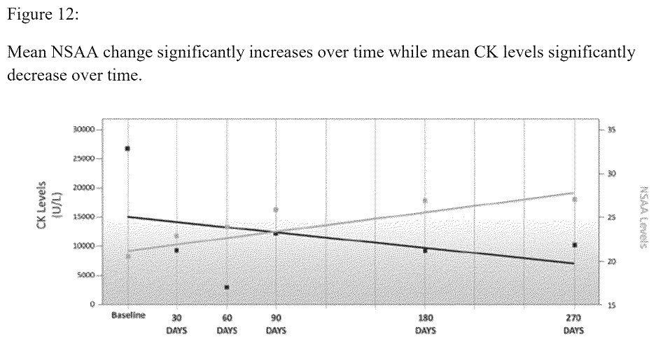

In addition to micro-dystrophin and CK levels, efficacy was measured by the following functional tests: Time to Rise from the floor, Ascend 4 steps, North Star Ambulatory Assessment (NSAA), Time to Rise Test, 4 Stairs Up Test, 10 Meter Timed Test (10 m), and 100 Meter Timed Test (100 m). The data is provided in Tables 5 and 6 below, and this data demonstrates consistent durable improvement at 9 months after administration of rAAVrh74.MHCK7.micro-dystrophin. The NSAA improvement over time is also provided in FIG. 12.

TABLE 5
NSAA Change from Baseline to Day 270

| | | | | | | | Change |
|---|---|---|---|---|---|---|---|
| | | | | | | | from |
| | | Day | Day | Day | Day | Day | Base- |
| Subject | Baseline | 30 | 60 | 90 | 180 | 270 | line |
| 1 | 18 | 22 | 24 | 23 | 25 | 26 | 8 |
| 2 | 19 | 21 | 23 | 25 | 27 | 27 | 8 |
| 3 | 26 | 28 | 28 | 30 | 30 | 28 | 2 |
| 4 | 19 | 20 | 20 | 25 | 25 | 27 | 8 |
| Mean | 20.5 | 22.75 | 23.75 | 25.75 | 26.75 | 27 | 6.5 |
| Improve- | | | | | | | |
| ment | | | | | | | |

TABLE 6
Change from Baseline to Day 270

| | Assess- | | Time to | 4 Stairs | 100 m | |
|---|---|---|---|---|---|---|
| Subject | ment | NSAA | Rise | Up | (sec) | 10 m |
| 1 | Baseline | 18 | 3.7 | 3.4 | 49.3 | 5.1 |
| | Day 270 | 26 | 3.0 | 2.3 | 43.2 | 4.3 |
| 2 | Baseline | 19 | 3.0 | 3.8 | 49.9 | 4.3 |
| | Day 180 | 27 | 3.7 | 2.6 | 48.6 | 3.9 |
| | Day 270 | 27 | 3.3 | 2.7 | 50.3 | |
| 3 | Baseline | 26 | 3.9 | 1.9 | 59.3 | 4.7 |
| | Day 180 | 30 | 3.4 | 1.8 | 48.4 | 4.1 |
| | Day 270 | 28 | 2.8 | 1.9 | 50.7 | |
| 4 | Baseline | 19 | 4.1 | 4.8 | 67.2 | 5.4 |
| | Day 90 | 25 | 2.3 | 2.2 | 50.7 | 4.4 |
| | Day 270 | 27 | 2.4 | 2.2 | 49.7 | |
| Aver- | Change | 6.5 point | .8 sec. | 1.2 sec. | 7.95 sec. | 14% |
| age | from | im- | im- | im- | im- | im- |
| | Baseline | prove- | prove- | prove- | prove- | prove- |
| | | ment | ment | ment | ment | ment |

### Safety Assessment

No serious adverse events (SAEs) were observed in the study. Three subjects had elevated gamma-glutamyl transferase (GGT) that resolved with increased steroids within a week and returned to baseline levels. There were no other clinically significant laboratory findings. Patients had transient nausea generally during the first week of therapy coincident with increased steroid dosing. This did not correlate with liver enzyme elevations or any other abnormality.

### Example 3

### Randomized Double-Blind Placebo Controlled Systemic Gene Delivery Phase I/IIa Clinical Trial

This is a randomized double-blind single-dose trial using rAAVrh74.MHCK7.micro-dystrophin for DMD subjects. The study includes twenty-four subjects ages 4 to 7 years old. Subjects are randomized to treatment or placebo at the time of enrollment. Twelve subjects receive intravenous rAAVrh74.MHCK7.micro-dystrophin vector (2×10¹⁴ vg/kg in approximately 10 mL/kg) and twelve subjects will receive 10 mL/kg placebo (lactated ringers). Placebo subjects will roll over to treatment which will be given in the same manner as the 12 previously treated subjects one year after the last treated subject is dosed. Subjects receive infusions of rAAV carrying micro-dystrophin or lactated ringers over approximately 1 hour. Pre and post-treatment (90 Day) needle muscle biopsies are done on gastrocnemius muscles.

The primary objective of this study is the assessment of the safety of intravenous administration of rAAVrh74.MHCK7.micro-dystrophin for DMD subjects via peripheral limb vein. Safety endpoints are assessed by changes in hematology, serum chemistry, urinalysis, immunologic response to rAAVrh74 and micro-dystrophin, and reported history and observations of symptoms. Dystrophin gene expression serves as a primary outcome measure along with safety. Quantification is carried out using validated immunofluorescence and immunoblot assays. A decrease in CK following gene therapy serves as a secondary outcome. Efficacy is measured by the following functional tests: Time to Rise, Ascend 4 steps, North Star Ambulatory Assessment (NSAA), 10 Meter Timed Test (10 m), 100 Meter Timed Test (100 m). Exploratory measures include hand-held dynamometry (HHD) for knee extensors and knee flexors, and elbow flexors and elbow extensors.

Inclusion criteria for the study are as follows:
Age of enrollment: between 4-7 years of age, inclusive.
Molecular characterization of the DMD gene with frameshift (deletion or duplication), or premature stop codon mutation between exons 18 to 58.
Indication of symptomatic muscular dystrophy: CK elevation>1000 U/L and Below mean percent predicted time on 100 meter walk test
Males of any ethnic group will be eligible.
Ability to cooperate with motor assessment testing.
Stable dose equivalent of oral corticosteroids for at least 12 weeks prior to screening and the dose is expected to remain constant (except for potential modifications to accommodate changes in weight) throughout the study.

Exclusion criteria for the study are as follows:
Active viral infection based on clinical observations.
Signs of cardiomyopathy, including echocardiogram with ejection fraction below 40%.
Serological evidence of HIV infection, or Hepatitis B or C infection.
Diagnosis of (or ongoing treatment for) an autoimmune disease.
Abnormal laboratory values considered clinically significant (GGT>3×ULN, bilirubin≥3.0 mg/dL, creatinine≥1.8 mg/dL, Hgb<8 or >18 g/Dl; WBC>18,500 per cmm), platelets≤50,000.
Concomitant illness or requirement for chronic drug treatment that in the opinion of the PI creates unnecessary risks for gene transfer.
Subjects with AAVrh74 or AAV8 antibody titers>1:400 as determined by ELISA immunoassay. If endpoint titer is positive at screening, testing may be repeated prior to exclusion.
Has a medical condition or extenuating circumstance that, in the opinion of the investigator, might compromise the subject's ability to comply with the protocol required testing or procedures or compromise the subject's wellbeing, safety, or clinical interpretability.
Severe infection (e.g., pneumonia, pyelonephritis, or meningitis) within 4 weeks before gene transfer visit (enrollment may be postponed).
Has received any investigational medication (other than corticosteroids) or exon skipping medications (including ExonDys 51®), experimental or otherwise, in the last 6 months prior to screening for this study.
Has had any type of gene therapy, cell based therapy (e.g. stem cell transplantation), or CRISPR/Cas9 therapy.
Family does not want to disclose patient's study participation with primary care physician and other medical providers.

### Efficacy Assessments

Dystrophin gene expression serves as a primary outcome measure along with safety. Quantification is carried out using validated immunofluorescence and immunoblot assays. A decrease in CK following gene therapy serves as a secondary outcome. In addition, efficacy is measured by the following functional tests: Time to Rise from the floor, Ascend 4 steps, North Star Ambulatory Assessment (NSAA), 10 Meter Timed Test (10 m), 100 Meter Timed Test (100 m)]. Exploratory measures include hand-held dynamometry (HHD) for knee extensors and knee flexors, and elbow flexors and elbow extensors.

Muscle biopsies with ultrasound guidance are used to quantify transgene expression comparing baseline to Day 90. The biopsies are carried out on the same muscle as the original biopsies but on the opposite leg. One year after all subjects have been dosed, placebo crossover subjects will restart the study timeline at visit 1. Placebo subjects will not have the following performed at the second baseline screening: Cardiac MRI and Muscle Biopsy. Placebo subjects undergo a muscle biopsy at Day 90 (total of 3 muscle biopsies). Frozen sections are stained for dystrophin using indirect immunofluorescence (IF). Full slide scanning is performed and micro-dystrophin intensity and percent positive fibers is quantified using validated image scanning and MuscleMap™ analysis algorithm. Muscle morphometrics are performed blinded including fiber size histograms. Blinded frozen muscle biopsy shavings are used to perform quantitative protein analysis for micro-dystrophin using a validated western blot method.

Muscle needle biopsies of the gastrocnemius muscle (unless deemed contraindicated in a specific subject by the PI, in which case the PI will select an alternative muscle to biopsy) are used to quantify micro-dystrophin expression.

### Efficacy Analyses

The primary efficacy endpoint is the change from Baseline to Day 90 in the quantity of micro-dystrophin protein expression as measured by Western blot of biopsied muscle tissue. Treatment group differences for the primary efficacy endpoint are assessed with an analysis of covariance (ANCOVA) model with treatment as the fixed factor and baseline value as the covariate. The Wilcoxon rank-sum test is performed as a supportive analysis. Change from Baseline in micro-dystrophin expression via immunofluorescence (IF) fiber intensity is analyzed similarly.

The supportive efficacy endpoints include change from Baseline to each scheduled assessment of Time to Rise from the floor, Ascend 4 steps, NSAA, Meter Timed Test (10 m), 100 Meter Timed Test (100 m), and change in CK. Exploratory measures include HHD for knee extensors and knee flexors, and elbow flexors and elbow extensors. Treatment group differences are assessed with ANCOVA model with treatment as the fixed factor and baseline value as the covariate. The Wilcoxon rank-sum test is performed as a supportive analysis.

### Example 4

The trials and studies described in Examples 2 and 3 above are alternatively carried out utilizing the rAAVrh74.MHCK7.micro-dystrophin construct set forth in SEQ ID NO: 9; as set forth in SEQ ID NO: 8, nucleotides 1-4977; or as set forth in SEQ ID NO: 6; nucleotides 56-5022.

### Example 5

Generation of the pAAV.MCK.Micro-Dystrophin Construct

The pAAV.MCK.micro-dystrophin plasmid was constructed by inserting the MCK expression cassette driving a codon optimized human micro-dystrophin cDNA sequence into the AAV cloning vector psub201 (Samulski et al., J. Virol. 61 (10): 3096-3101). A muscle-specific regulatory element was included in the construct to drive muscle-specific gene expression. This regulatory element comprised the mouse MCK core enhancer (206 bp) fused to the 351 bp MCK core promoter (proximal). After the core promoter, the construct comprises the 53 bp endogenous mouse MCK Exon1 (untranslated) for efficient transcription initiation, followed by the SV40 late 16S/19S splice signals (97 bp) and a small 5′UTR (61 bp). The intron and 5′ UTR was derived from plasmid pCMVβ (Clontech). The micro-dystrophin cassette has a consensus Kozak immediately in front of the ATG start and a small 53 bp synthetic polyA signal for mRNA termination. The human micro-dystrophin cassette contains the (R4-R23/Δ71-78) domains as previously described by Harper et al. Nat. Med. 8 (3): 253-61, 2002

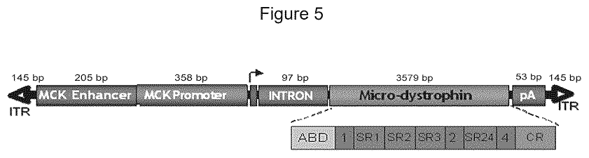

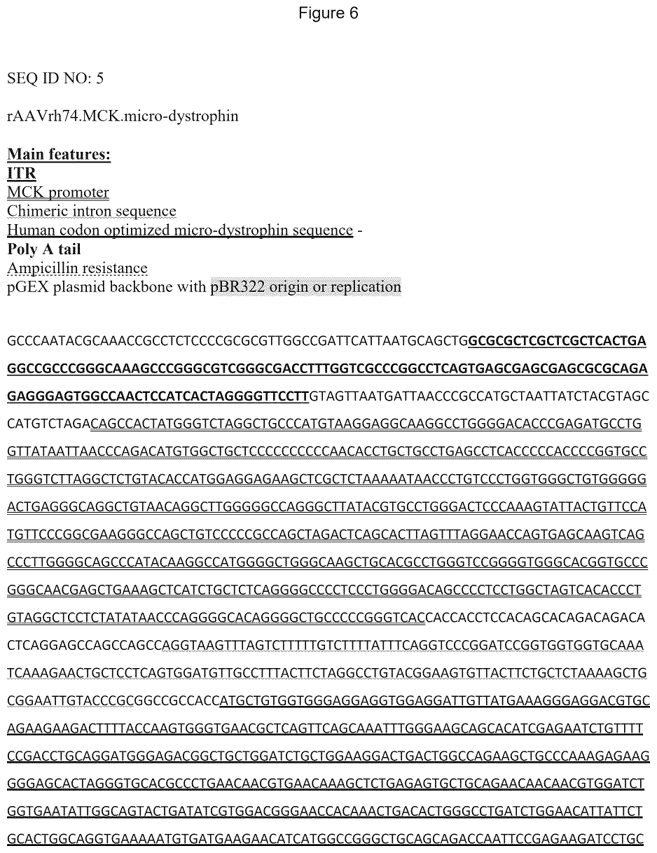

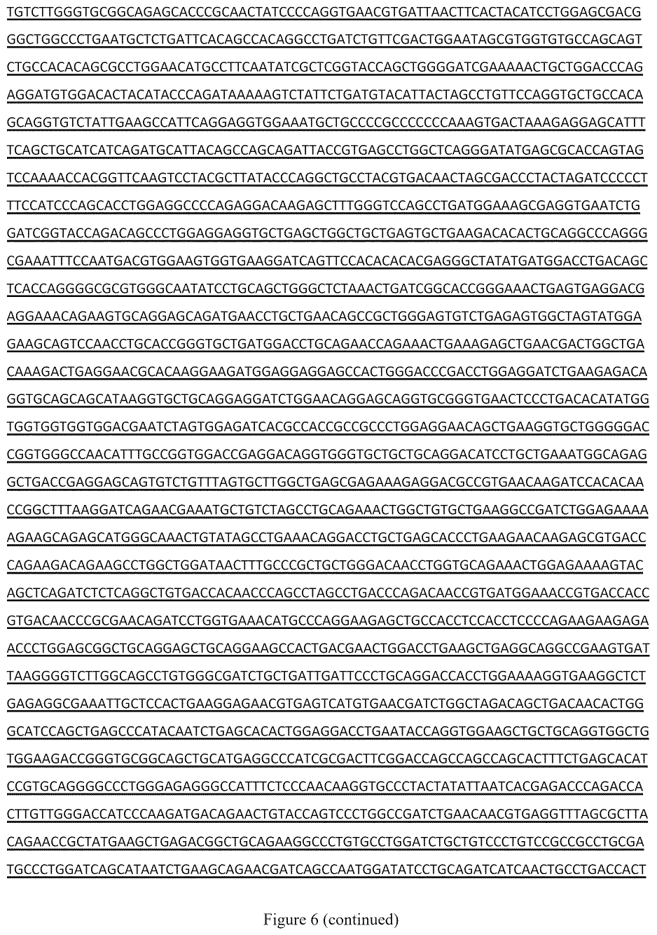

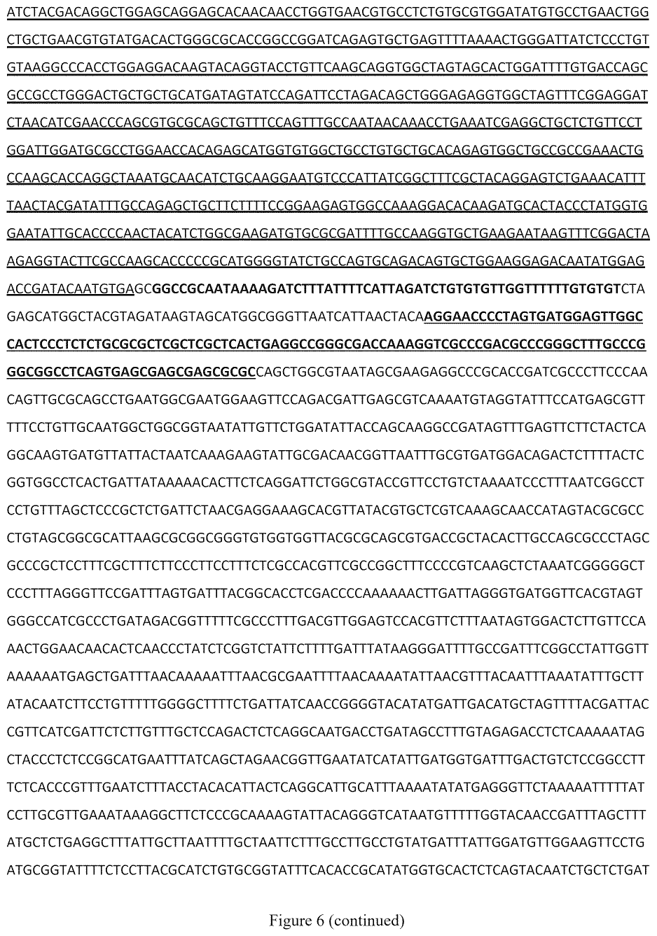

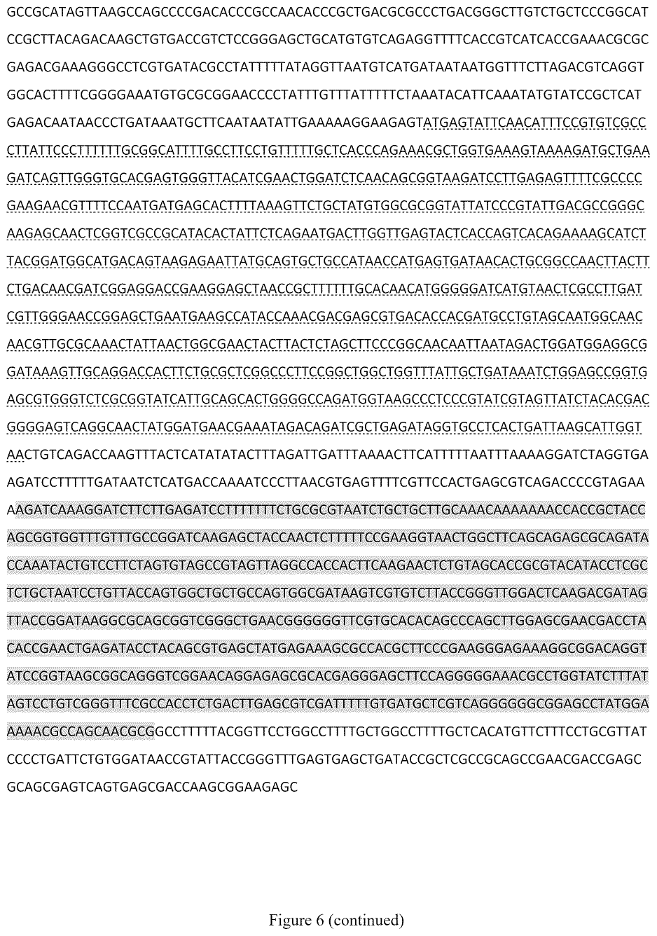

The pAAV.MCK.micro-dystrophin plasmid contained the human micro-dystrophin cDNA expression cassette flanked by AAV2 inverted terminal repeat sequences (ITR) (see FIG. 5). This sequence was encapsidated into AAVrh.74 virions. The molecular clone of the AAVrh.74 serotype was cloned from a rhesus macaque lymph node and is described in Rodino-Klapac et al. Journal of Tran. Med. 45 (2007).

### REFERENCES

1. Hoffman, E. P., Brown, R. H., Jr. & Kunkel, L. M. Dystrophin: the protein product of the Duchenne muscular dystrophy locus. Cell 51, 919-928 (1987).
2. Straub, V. & Campbell, K. P. Muscular dystrophies and the dystrophin-glycoprotein complex. Curr Opin Neurol 10, 168-175 (1997).
3. Sacco, A., et al. Short telomeres and stem cell exhaustion model Duchenne muscular dystrophy in mdx/mTR mice. Cell 143, 1059-1071 (2010).
4. Wallace, G. Q. & McNally, E. M. Mechanisms of muscle degeneration, regeneration, and repair in the muscular dystrophies. Annu Rev Physiol 71, 37-57 (2009).
5. Zhou, L. & Lu, H. Targeting fibrosis in Duchenne muscular dystrophy. J Neuropathol Exp Neurol 69, 771-776 (2010).
6. Desguerre, I., et al. Endomysial fibrosis in Duchenne muscular dystrophy: a marker of poor outcome associated with macrophage alternative activation. J Neuropathol Exp Neurol 68, 762-773 (2009).
7. DiPrimio, N., McPhee, S. W. & Samulski, R. J. Adeno-associated virus for the treatment of muscle diseases: toward clinical trials. Curr Opin Mol Ther 12, 553-560 (2010).
8. Mendell, J. R., et al. Sustained alpha-sarcoglycan gene expression after gene transfer in limb-girdle muscular dystrophy, type 2D. Ann Neurol 68, 629-638 (2010).
9. Mendell, J. R., et al. Limb-girdle muscular dystrophy type 2D gene therapy restores alpha-sarcoglycan and associated proteins. Ann Neurol 66, 290-297 (2009).
10. Mendell, J. R., et al. A phase 1/2a follistatin gene therapy trial for becker muscular dystrophy. Molecular therapy: the journal of the American Society of Gene Therapy 23, 192-201 (2015).
11. Carnwath, J. W. & Shotton, D. M. Muscular dystrophy in the mdx mouse: histopathology of the soleus and extensor digitorum longus muscles. J Neurol Sci 80, 39-54 (1987).
12. Coulton, G. R., Morgan, J. E., Partridge, T. A. & Sloper, J. C. The mdx mouse skeletal muscle myopathy: I. A histological, morphometric and biochemical investigation. Neuropathol Appl Neurobiol 14, 53-70 (1988).
13. Cullen, M. J. & Jaros, E. Ultrastructure of the skeletal muscle in the X chromosome-linked dystrophic (mdx) mouse. Comparison with Duchenne muscular dystrophy. Acta Neuropathol 77, 69-81 (1988).
14. Dupont-Versteegden, E. E. & McCarter, R. J. Differential expression of muscular dystrophy in diaphragm versus hindlimb muscles of mdx mice. Muscle Nerve 15, 1105-1110 (1992).
15. Stedman, H. H., et al. The mdx mouse diaphragm reproduces the degenerative changes of Duchenne muscular dystrophy. Nature 352, 536-539 (1991).
16. Deconinck, A. E., et al. Utrophin-dystrophin-deficient mice as a model for Duchenne muscular dystrophy. Cell 90, 717-727 (1997).
17. Grady, R. M., et al. Skeletal and cardiac myopathies in mice lacking utrophin and dystrophin: a model for Duchenne muscular dystrophy. Cell 90, 729-738 (1997).
18. Love, D. R., et al. An autosomal transcript in skeletal muscle with homology to dystrophin. Nature 339, 55-58 (1989).
19. Tinsley, J. M., et al. Primary structure of dystrophin-related protein. Nature 360, 591-593 (1992).
20. Tinsley, J., et al. Expression of full-length utrophin prevents muscular dystrophy in mdx mice. Nat Med 4, 1441-1444 (1998).
21. Squire, S., et al. Prevention of pathology in mdx mice by expression of utrophin: analysis using an inducible transgenic expression system. Hum Mol Genet 11, 3333-3344 (2002).
22. Rafael, J. A., Tinsley, J. M., Potter, A. C., Deconinck, A. E. & Davies, K. E. Skeletal muscle-specific expression of a utrophin transgene rescues utrophin-dystrophin deficient mice. Nat Genet 19, 79-82 (1998).
23. Zhou, L., et al. Haploinsufficiency of utrophin gene worsens skeletal muscle inflammation and fibrosis in mdx mice. J Neurol Sci 264, 10⁶-111 (2008).
24. Gutpell, K. M., Hrinivich, W. T. & Hoffman, L. M. Skeletal Muscle Fibrosis in the mdx/utrn+/−Mouse Validates Its Suitability as a Murine Model of Duchenne Muscular Dystrophy. PloS one 10, e0117306 (2015).
25. Rodino-Klapac, L. R., et al. Micro-dystrophin and follistatin co-delivery restores muscle function in aged DMD model. Human molecular genetics 22, 4929-4937 (2013).
26. Nevo, Y., et al. The Ras antagonist, farnesylthiosalicylic acid (FTS), decreases fibrosis and improves muscle strength in dy/dy mouse model of muscular dystrophy. PloS one 6, e18049 (2011).
27. Rodino-Klapac, L. R., et al. A translational approach for limb vascular delivery of the micro-dystrophin gene without high volume or high pressure for treatment of Duchenne muscular dystrophy. J Transl Med 5, 45 (2007).
28. Mulieri, L. A., Hasenfuss, G., Ittleman, F., Blanchard, E. M. & Alpert, N. R. Protection of human left ventricular myocardium from cutting injury with 2,3-butanedione monoxime. Circ Res 65, 1441-1449 (1989).
29. Rodino-Klapac, L. R., et al. Persistent expression of FLAG-tagged micro dystrophin in nonhuman primates following intramuscular and vascular delivery. Molecular therapy: the journal of the American Society of Gene Therapy 18, 109-117 (2010).
30. Grose, W. E., et al. Homologous recombination mediates functional recovery of dysferlin deficiency following AAV5 gene transfer. PloS one 7, e39233 (2012).
31. Liu, M., et al. Adeno-associated virus-mediated microdystrophin expression protects young mdx muscle from contraction-induced injury. Mol Ther 11, 245-256 (2005).
32. Harper, S. Q., et al. Modular flexibility of dystrophin: implications for gene therapy of Duchenne muscular dystrophy. Nature medicine 8, 253-261 (2002).
33. Rodino-Klapac, L. R., et al. Persistent expression of FLAG-tagged micro dystrophin in nonhuman primates following intramuscular and vascular delivery. Mol Ther 18, 109-117 (2010).
34. Salva, M. Z., et al. Design of tissue-specific regulatory cassettes for high-level rAAV-mediated expression in skeletal and cardiac muscle. Mol Ther 15, 320-329 (2007).
35. Sondergaard, P. C., et al. AAV.Dysferlin Overlap Vectors Restore Function in Dysferlinopathy Animal Models. Annals of clinical and translational neurology 2, 256-270 (2015).
36. De, B. P., et al. High levels of persistent expression of alpha1-antitrypsin mediated by the nonhuman primate serotype rh. 10 adeno-associated virus despite preexisting immunity to common human adeno-associated viruses. Mol Ther 13, 67-76 (2006).
37. Rodino-Klapac, L. R., et al. A translational approach for limb vascular delivery of the micro-dystrophin gene without high volume or high pressure for treatment of Duchenne muscular dystrophy. Journal of translational medicine 5, 45 (2007).
38. Bulfield et al., X chromosome-linked muscular dystrophy (mdx) in the mouse. Proc Natl Acad Sci USA. 1984; 81 (4): 1189-1192.
39. Sicinski et al., The molecular basis of muscular dystrophy in the mdx mouse: a point mutation. Science. 1989 30; 244 (4912): 1578-80

1. A composition comprising a polynucleotide comprising the nucleotide sequence of SEQ ID NO: 9.

2. The composition of claim 1, wherein the composition comprises a recombinant adeno-virus associated (rAAV) comprising the polynucleotide comprising the nucleotide sequence of SEQ ID NO: 9.

3.-10. (canceled)

11. A method of expressing a microdystrophin gene in a cell comprising contacting the cell with a recombinant adeno-virus associated (rAAV) serotype rh.74 comprising a polynucleotide comprising the nucleotide sequence of SEQ ID NO: 1 operably linked to an MHCK7 promoter.

12. The method of claim 11, wherein the MHCK7 promoter comprises the nucleotide sequence of SEQ ID NO: 7.

13. A method of decreasing serum CK levels in a subject in need thereof, the method comprising administering to the subject a recombinant adeno-virus associated (rAAV) serotype rh.74 comprising a polynucleotide comprising the nucleotide sequence of SEQ ID NO: 1 operably linked to an MHCK7 promoter.

14. A method of increasing microdystrophin positive fibers in muscle tissue of a subject comprising administering to the subject a recombinant adeno-virus associated (rAAV) serotype rh.74 comprising a polynucleotide comprising the nucleotide sequence of SEQ ID NO: 1 operably linked to an MHCK7 promoter.

15. A method of treating Duchenne muscular dystrophy in a human subject in need thereof comprising administering an recombinant adeno-virus associated (rAAV) serotype rh.74 comprising a polynucleotide comprising the nucleotide sequence of SEQ ID NO: 1 operably linked to a promoter sequence of SEQ ID NO: 7, wherein the rAAV is administered intravenously at a dose of 2×10¹⁴ vg/kg, and wherein the human subject has a AAVrh.74 antibody titer<1:400 as determined by ELISA immunoassay before administration of the rAAV serotype rh.74 to the subject.

16. The method of claim 15, wherein the human subject is between three months and seven years of age.

17. The method of claim 15, wherein the human subject is between four years of age and seven years of age.

18. The method of claim 15, wherein the human subject is between three months of age and three years of age.

19. The method of claim 15, wherein the dose of the rAAV of serotype rh.74 is determined by utilizing a super-coiled DNA standard.

20. The method of claim 15, wherein the dose of the rAAV is administered in about 10 mL/kg.

21. The method of claim 15, wherein the rAAV is administered by infusion over approximately one hour.

22. The method of claim 15, wherein the level of microdystrophin gene expression in a muscle cell of the subject is increased after administration of the rAAV as compared to the level of micro-dystrophin gene expression before administration of the rAAV.

23. The method of claim 15, wherein the level of microdystrophin protein is increased by at least 72% after administration of rAAV compared to the level of micro-dystrophin before administration of rAAV.

24. The method of claim 15, wherein the serum CK level in the subject is decreased after administration of the rAAV as compared to the serum CK level before administration of the rAAV.

25. The method of claim 15, wherein the number of microdystrophin positive fibers in a muscle tissue of the subject is increased after administration of the rAAV as compared to the number of micro-dystrophin positive fibers before administration of the rAAV.

26. The method of claim 15, wherein the level of alpha-sarcoglycan or beta-sarcoglycan in the subject is increased after administration of the rAAV as compared to the level of alpha-sarcoglycan or beta-sarcoglycan before administration of the rAAV.

27. The method of claim 15, wherein the serum CK level in the subject is decreased after administration of the rAAV as compared to the serum CK level before administration of the rAAV by a percentage level selected from the group consisting of:
a) at least 78% by 90, 180, or 270 days after the administration;
b) at least 46, 55, 56, 57, 58, 59, 60, 61, 62, 63, 64, 65, 66, 67, 68, 69, 70, or 85% by 270 days after the administration;
c) at least 72, 73, 74, or 95% by 180 days after the administration;
d) at least 87, 88, 93 or 95% by 90 days after the administration;
e) at least 70% by 270 days after the administration;
f) 70 to 95% by 90, 180, or 270 days after the administration; and
g) at least 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95% by 90, 180, or 270 days after the administration.

28. The method of claim 15, wherein disease progression in the subject is delayed after administration of the rAAV as measured by one or more of: the six-minute walk test, time to rise, ascend 4 steps, ascend and descend 4 steps, North Star Ambulatory Assessment (NSAA), 10 meter timed test, 100 meter timed test, hand-held dynamometry (HHD), Timed Up and Go, and/or Gross Motor Subtest Scaled (Bayley-III) score.
